(19) United States
(12) Patent Application Publication
(10) Pub. No.: US 2024/0110163 A1
(43) Pub. Date: Apr. 4, 2024

(54) CRISPR-ASSOCIATED BASED-EDITING OF THE COMPLEMENTARY STRAND

(71) Applicant: Wageningen Universiteit, Wageningen (NL)

(72) Inventors: John VAN DER OOST, Renkum (NL); Wen Ying WU, Wageningen (NL); Ricardo VILLEGAS WARREN, Wageningen (NL)

(21) Appl. No.: 18/273,501

(22) PCT Filed: Jan. 28, 2022
(86) PCT No.: PCT/NL2022/050042

§ 371 Date: Jul. 20, 2023

(30) Foreign Application Priority Data
Jan. 29, 2021 (EP) 21154421.8
Apr. 1, 2021 (EP) 21166717.5

(51) Int. Cl. C12N 9/22; C12N 9/78; C12N 15/63

(52) U.S. Cl. CPC C12N 9/22; C12N 9/78; C12N 15/63; C12N 2310/20; C12Y 305/04002; C12Y 305/04005

(57) ABSTRACT

The invention relates to methods for clustered regularly interspaced short palindromic repeat (CRISPR)-mediated A to G and/or C to T editing of the guide-complementary strand of a double stranded target DNA. The invention further relates to a nucleotide molecule encoding said CRISPR-mediated base editing system, to an expression vector comprising the nucleotide molecule, and to a cell comprising the expression vector. The invention further relates to an isolated Cas nuclease that allows A to G and/or C to T editing of the complementary strand of a double stranded target nucleic acid.

### RELATED APPLICATIONS

This application is a 371 National Stage application of International Application No. PCT/NL2022/050042, filed Jan. 28, 2022, which claims priority from EP21154421.8, filed on Jan. 29, 2021 and EP21133717.5, filed Apr. 1, 2021, which are herein incorporated by reference in their entireties.

### INCORPORATION OF SEQUENCE LISTING

A sequence listing containing the file named “VONL028US ST25.txt” which is 61.9 kilobytes (measured in MS-Windows®) and created on Jul. 13, 2023, and comprises 229 sequences, is incorporated herein by reference in its entirety.

FIELD: The invention relates to CRISPR based gene manipulation and to CRISPR endonucleases that provide for methods of expression control and gene editing in cells.

### 1 INTRODUCTION

In the last decade, genome editing by CRISPR-Cas nucleases has taken the world by storm, offering an effective, precise, and efficient way of genome editing (Mohanraju et al., 2016. Science 353, aad5147; Wu et al., 2018. Nature Chem Biol 14: 642; Anzalone et al., 2020. Nature Biotech 38, 824-844). On the one hand, gene disruption relies on generating a double strand DNA break in the gene of interest, after which an error-prone repair of the broken strand occurs through the non-homologous end joining NHEJ) system, which appears abundant in eukaryotes but rare in prokaryotes (Bertrand et al., 2019. Mol Microbiol 111: 1139-1151; Chang et al., 2017. Nature Reviews Mol Cell Biol 18: 495). For precise genome editing, on the other hand, a repair template must be delivered to the cell, requiring a homology-directed repair (HDR) system, the availability of which can substantially differ from one cell type to the other (Verma and Greenberg, 2016. Genes Develop 30: 1138-1154). It is important to note, however, that not all genome editing applications require large modifications, e.g. repairing a single nucleotide polymorphism (SNP) can be accomplished by a specific single nucleotide substitution (Rees and Liu, 2018. Nature Rev Genet 19: 770-788). In addition, apart from repairing SNPs, single nucleotide mutations can also introduce a premature stop codon for generating gene knockouts (Komor et al., 2016. Nature 533: 420-424; Kuscu et al., 2017. Nature Methods 14: 710.

To circumvent the need to deliver a repair template for each single nucleotide mutation, base editors were developed. Synthetic CRISPR-associated base editor allows for RNA-guided, targeted nucleotide substitutions (C to T) on the non-target strand. The first base editor that was developed consisted of a chimeric construct of a Cas9, a cytidine deaminase and an uracil glycosylase inhibitor (UGI) (Komor et al., 2016. Nature 533: 420-424; Nishida et al., 2016. Science 353: aaf8729; Banno et al., 2018. Nature Microbiol 3: 423-429). After gRNA-guided recognition, the catalytically inactive variant of Cas9 (D10A and H840A) also known as dead Cas9 (dCas9), which is unable to cleave dsDNA, targets and unwinds its dsDNA target. After DNA unwinding, the cytidine deaminase catalyzes the deamination of cytidine to uridine (C to U) in the displaced non-target strand, which leads to replacement by thymidine after replication, hence C to T. In addition, the role of the UGI domain is to inhibit the uracil glycosylase enzyme and as such preventing base excision repair, thereby increasing the C to T editing efficiency.

Initially, dCas9 was used, because the role of Cas9 was just to specifically bind and unwind of a selected dsDNA target. In subsequent base editor designs, however, nickase Cas9 (nCas9) variants are often used instead as it was found that a break in the target strand results in elevated base editing efficiencies, most likely by promoting mismatch repair in which the edited non-target strand serves as template, resulting in the desired overall base pair substitution: C-G via T-G to T-A (Komor et al., 2016. Nature 533: 420-424; Nishida et al., 2016. Science 353: aaf8729).

Up until now, several designs of Cas9 C to T base editors have been generated to reduce the base editing range within the protospacer (base editing window) and to increase the base editing efficiency (Kim et al., 2017. Nature Biotech 35: 371-376; Komor et al., 2017. Science Advances 3: eaao4774; Thuronyi et al., 2019. Nature Biotech 37: 1070-1079). In addition, also a dCas12a C to T base editor has been created to expand the base editing toolbox, allowing for targeting of sequences downstream a 5′ (T)TTV PAM instead of sequences upstream a 3′ NGG PAM in case of Cas9 (Kleinstiver et al., 2019. Nature Biotech 37: 276-282; Li et al., 2018. Nature Biotech 36: 324-327). Cas9 and Cas12a base editors also differ with respect to their editing windows. Base editing positions are numbered relative to the PAM-distal end and the PAM proximal end of the protospacer for Cas9 and Cas12a, respectively. For example, the NGG PAM sequence of Cas9 is numbered 21 to 23 and the (T)TTV PAM sequence of Cas12a is numbered −4 to −1. Cas9 and Cas12a base editors target C's in positions 3-8 and 8-13, respectively (Komor et al., 2016. Nature 533: 420-424; Nishida et al., 2016. Science 353: aaf8729; Kim et al., 2017. Nature Biotech 35: 371-376; Kleinstiver et al., 2019. Nature Biotech 37: 276-282; Li et al., 2018. Nature Biotech 36: 324-327; Tan et al., 2019. Nature Commun 10: 1-10).

Next to the aforementioned CRISPR-associated C to T base editor (CBE), also an A to G base editor (ABE) has been made by fusing an adenine deaminase domain to Cas9, followed by directed evolution and engineering (Gaudelli et al., 2017. Nature 551: 464-471; Lapinaite et al., 2020. Science 369: 566-571). These base editors convert targeted A-T base pairs to G-C, with the initial deaminase-based editing occurring on the displaced, non-target strand.

A major limitation of the afore mentioned CBEs and ABEs is their dependence on a correct spacing between an appropriate PAM motif, such as 3′-NGG for SpCas9 and 5′-(T)TTV for Cas12a, and the position of the desired edit. This implies that the current set of Cas9- and Cas12a-associated CBEs and ABEs does not allow all desired edits to be made. There is thus a need for Cas-associated base editors with different features, such as PAM, editing window and the capacity to edit the target strand in addition to the non-target strand of the target DNA.

### 2 BRIEF DESCRIPTION OF THE INVENTION

The present invention surprisingly provides methods and means for base editing, in particular for making A to G and C to T modifications on the non-displaced, complementary strand of the target DNA. As is known to a person skilled in the art, the complementary strand of a double stranded nucleic acid is the strand that is complementary to the spacer sequence in the guide RNA. These A to G modifications equal T to C modifications on the displaced, non-complementary strand of the target DNA, while the C to T modifications equal G to A modifications on the displaced, non-complementary strand of the target DNA, thereby expanding the range of edits that can be made using base editing.

The invention therefore provides a method for base-editing a double stranded target nucleic acid (dstna) in a cell, the method comprising the step of providing the cell with a clustered regularly interspaced short palindromic repeat (CRISPR)-based editing system comprising a guide ribonucleic acid (gRNA) of which a part is complementary to a nucleic acid strand of a dstna, and a cleavage-deficient CRISPR-associated (Cas) nuclease that is fused to a deaminase, preferably an adenine deaminase or a cytidine deaminase, whereby the editing system provides A to G editing of the complementary strand and/or the non-complementary strand of the dstna, or C to T editing of the complementary strand and/or the non-complementary strand of the dstna, respectively. Said adenine deaminase may also be combined with a cytidine deamination enzyme, either as tandem fusions to the N-terminus or C-terminus of the cleavage-deficient Cas nuclease, or whereby the adenine deaminase is fused to one terminus, and the cytidine deamination enzyme is fused to the other terminus.

The deaminase, preferably a cytidine or an adenine deamination enzyme, or both an adenine deamination enzyme and a cytidine deamination enzyme, are preferably fused to the C-terminus of the cleavage-deficient Cas nuclease.

It was surprisingly found that a Cas nuclease which lacks certain domains, and/or which is of a limited size, facilitates A to G editing, or C to T editing, of the complementary strand of the gRNA, in addition to A to G or C to T editing of the non-complementary strand of the dstna.

To accomplish A to G or C to T editing of the complementary strand, said Cas nuclease preferably lacks at least part of a recognition lobe, termed Rec1 and Rec2 domains, when compared to other Cas nucleases, especially Cas12a and Cas12b nucleases, preferably most of the recognition lobe.

As an alternative, or in addition, said Cas nuclease preferably comprises 300-800 amino acid residues, preferably less than 650 amino acid residues, less than 600 amino acid residues, less than 550 amino acid residues, less than 500 amino acid residues, less than 450 amino acid residues, less than 400 amino acid residues. A cleavage-deficient Cas nuclease may comprise 695 or 422 amino acid residues.

A further preferred cleavage-deficient Cas nuclease multimerizes upon binding to the (one) gRNA in the CRISPR-based editing system, preferably dimerizes. Said multimerization, preferably dimerization, may be inducible, for example by the presence of a dimerization domain. Said dimerization domain may be a chemically-sensitive dimerization domain or a light-sensitive dimerization domain, as will be explained herein below. Said cleavage-deficient Cas nuclease preferably forms a homomeric, preferably homodimeric, molecule.

Said cleavage-deficient Cas nuclease preferably is a naturally occurring nuclease such as a naturally occurring C2c4 nuclease, or a naturally occurring Cas12F nuclease, or an altered C2c4 nuclease or Cas12F nuclease, e.g. having one or more mutations that abolish at least double stranded cleavage activity.

Said cleavage-deficient Cas nuclease may be fused to a deaminase, such as a cytidine deaminase and/or an adenine deaminase, preferably at the C-terminus of the cleavage-deficient Cas nuclease. In a preferred embodiment, a loop structure at the C-terminal region of the deaminase, preferably an adenine deaminase, is duplicated at the N-terminal region of the deaminase, and a C-terminal helix structure is duplicated and invertedly inserted at the N-terminus of the deaminase.

Said cleavage-deficient Cas nuclease preferably is fused to a deaminase through a linker sequence.

The invention further provides a nucleotide molecule encoding a Cas nuclease that is fused to at least a deaminase allowing A to G editing or C to T editing, of the complementary strand of the ds target nucleic acid, as described herein.

The invention further provides an expression vector comprising the nucleotide molecule of the invention, under control of a suitable expression promoter. Said expression vector may further comprise a nucleotide sequence comprising an expression promoter and a sequence under the control of the promoter encoding a gRNA, said gRNA comprising a nucleotide sequence which recognises a target locus of interest.

The invention further provides a cell comprising an expression vector according to the invention.

The invention further provides an isolated Cas nuclease that is fused to a deaminase, allowing A to G editing or C to T editing of the complementary strand of a double stranded target nucleic acid, as described herein. Said isolated Cas nuclease preferably comprises an adenine or cytidine deamination enzyme which is fused to C-terminal end of the Cas nuclease, optionally wherein a cytidine deamination enzyme is fused to the N-terminal end of the Cas nuclease and an adenine deamination enzyme is fused to the C-terminal end.

### 3 FIGURE LEGENDS

FIG. 1. C to T base editing by MmuBE1_E1. (A) Schematic of MmuBE_E1 gene. (B) Schematic of base editing process by MmuBE_E1. MmuBE_E1 recognizes a 5′ TTN PAM and binds to its target. Once an R-loop is formed, CDA deaminases a C to a U. Then mismatch repair and DNA replication generate a dsDNA containing a T instead of a C. (C) Overview of the C-tile targets used to characterize the editing window of MmuBE_E1. The wildtype sequence contains a C at position 3 and serves as an internal standard for base editing. C-tile 1 to C-tile 6 plasmids contain six consecutive C's in the sequence and shifts three position toward 3′ end until position 20 is reached. (D) Deep sequencing results of MmuBE_E1 targeting the C-tile plasmids after 16 hours. Data from plasmids of uneven and even numbers were fused for easier data overview corresponding to ‘Merged 1, 3, 5’ and ‘Merged 2, 4, 6’, respectively. Y-axis represent base edited plasmids in % of the whole plasmid population and x-axis represent the C position within the protospacer.

FIG. 2. Schematic of different MmuBEs. All MmuBE consists, from left to right, of a dMmu, linker, cytidine deaminase, and UGI. E. coli (FIG. 2A) and H. sapiens (FIG. 2B) Mmu base editors consist of genes harmonized or optimized for E. coli and H. sapiens, respectively. Linkers are indicated with a number, representing the amino acid length. In addition, MmuBE_E and MmuBE_H also have an LVA degradation tag or nuclear localization sequences (NLS), respectively, as indicated.

FIG. 3. Silencing and base editing by various MmuBEs. (A) Schematic of GFP silencing by MmuBE. (B) GFP silencing by various MmuBEs. Y-axis represents relative GFP fluorescence in % where negative control frameshift dMmu (FSdMmu) was used as 100%. X-axis represent the different MmuBEs tested. (C) Base editing targets consisting of a C on every first, second and third position of each trinucleotide. These plasmids were names C1, C2 and C3 motif, respectively. (D) Heat map representing % of base edited C's using different variants of E. coli MmuBEs (MmuBE_E). Data was obtained by fusion C1, C2 and C3 motif data. (E) Heat map representing % of base edited C's using different variants of H. sapiens MmuBEs (MmuBE_H). Data was obtained by fusion C1, C2 and C3 motif data.

FIG. 4. Base editing in S. cerevisiae using MmuBE_S. (A) Schematic of MmuBE_S gene. (B) Experimental set-up for testing base editing in S. cerevisiae. ade2 gene in S. cerevisiae is targeted by MmuBE_S and if successfully base edited, premature STOP codon is created (red line). If ADE2 is not expressed in the absence of adenine, a red pigment accumulates into the cells and the yeast colony will appear red on the plate. Red colonies were picked, ade2 region amplified and sequenced. (C) Schematic of base editing workflow for S. cerevisiae. Cells expressing the MmuBE_S are cultured in flasks for 24 hours, plated to distinguish between edited cells (grey) and non-edited cells (white) and sequenced. (D) Sequencing results of three MmuBE_S targets, ADE2_1, ADE2_2, ADE2_3. Numbers indicate number of red colonies that were base edited compared to the number of colonies sent for sequencing. Arrows indicate position where base editing took place.

FIG. 5. Engineering of TadA8e to TadA8e_eng. An α-helix sequence at the C-terminus end of TadA8e was inverted and positioned at the N-terminus linked by a loop (yellow). Amino acids serine (S) and glutamic acid (E) were removed from the N-terminus to better link to the loop to the N-terminus and keep the amino acids glutamine-valine (QV) at the end of the loop.

FIG. 6. Base editing using Cas12u1 ABEs. (A) Schematics of various Cas12u1 ABE constructs (B). Protospacers used as targets for tested A to G base editing. A1, A2 and A3 motif plasmids contain an adenine (A; underlined) starting at every first, second or third nucleotide of the protospacer, respectively. (C) Fused A to G editing data obtained from A1, A2 and A3 motifs after 48 hours. Numbers indicate % of population that contains A to G editing at the given nucleotide position. An A is always present in position four in all three A motif plasmids, thereby the highest value of base editing was taken to represent position 4. (D) Fused T to C editing data obtained from A1, A2 and A3 motifs after 48 hours. Numbers indicate % of population that contains A to G editing at the given nucleotide position. A T is always present in position twenty-three in all three A motif plasmids, thereby the highest value of base editing was taken to represent position twenty-three.

FIG. 7. Base editing using Cas12f1 ABEs. (A) Schematic of Cas12f1 ABE construct. (B). Protospacers used as targets for tested A to G base editing. C1, C2, C3 and A1 motif plasmids were used and cover in total 13 positions on the 20 nt protospacer except for position 3, 5, 6, 11, 12, 15 and 18. (C) % A to G base editing of C1, C2, C3 and A2 motif plasmids after 72 hours. Numbers indicate % A to G editing at the given nucleotide position. (D) % T to C base editing of C1, C2, C3 and A2 motif plasmids after 72 hours. Numbers indicate % population containing T to C editing at the given nucleotide position.

FIG. 8. Base editing using Cas12f1 ABEs. (A) Schematics of AsCas12f1 CBE constructs. (B) Protospacers used as targets for tested C to T base editing. (C) Merged C to T editing data obtained from C1, C2 and C3 motif after 72 hours. Numbers indicate % of population that contains C to T editing at the given nucleotide position. A C is always present in position four in all three C motif plasmids, thereby the highest value of base editing was taken to represent position 4. (D) Merged C to T editing data obtained d from A1, A2 and A3 motifs after 72 hours. Numbers indicate % of population that contains C to T editing at the given nucleotide position. (E) Merged G to A editing data obtained from A1, A2 and A3 motifs after 72 hours. Numbers indicate % of population that contains G to A editing at the given nucleotide position.

FIG. 9. Cas12f1 CBEs targeting DUX4. Three protospacers were tested targeting a 5′-ATTA or 5′-TTTA PAM. Numbers indicate % of population that contains C to T editing at the given nucleotide position.

FIG. 10. Amino acid sequences of C to T editing constructs. Indicated are the amino acid sequences of Cas12f1 CBE1 and Cas12f1 CBE2. Cas12f1 amino acid sequences are indicated as simple text, linker sequences are underlined, cytidine deaminase amino acid sequences of APOBEC (Cas12f1 CBE1) and CDA (Cas12f1 CBE2) are in bold and italics; uracil glycosylase inhibitor (UGI) amino acid sequences are in bold, and nuclear localization sequences are in italics.

FIG. 11. C to T editing by Cas12f1-CBEs. (A) Schematic of Cas12f1-CBEs consisting of a dCas12f1, linker, cytidine deaminase, UGI and a nuclear localization signals (NLS), as indicated. The nucleotide sequence of E. coli Cas12f1-CBEs were codon harmonized or codon optimized for expression in E. coli, except for Has rAPOBEC1, which refers to a gene that was codon optimized for expression in Homo sapiens. Linkers are indicated with a number, representing the amino acid length. (B) Tile protospacers used for C to T editing by Cas12f1-CBEs. Numbers indicate position on protospacer.

FIG. 12. Editing on C/G tile by Cas12f1-CBEs. Heatmap of merged data obtained from C or G tiling. Average and standard deviation (stand. dev) are calculated for positions for which editing data was obtained using more than one tile plasmid. (A) results from C-tile editing by Cas12f1-CBE1 and Cas12f1-CBE2. (B) results from G-tile editing by Cas12f1-CBE1 and Cas12f1-CBE2.

### 4 DETAILED DESCRIPTION OF THE INVENTION

### 4.1 Definitions

The invention encompasses methods of modifying a genomic locus of interest to change gene expression in a cell by introducing into the cell any of the compositions described herein. This may include medical uses in humans for therapeutic or non-therapeutic purposes. Furthermore, any of the methods described herein may be applied in vitro and ex vivo. For therapeutic purposes, these methods may be termed gene- or genome editing, or gene therapy. The invention also encompasses methods of modifying genomic loci for non-medical uses in animals, plants, algae, fungi, and prokaryotes including bacteria and archaea.

The term “base-editing”, as is used herein, refers to the conversion of one target base or base pair into another, for example A:T to G:C, or C:G to T:A, without requiring the creation and repair of double strand breaks. Base editing may be achieved with the help of DNA and RNA base editors that allow the introduction of point mutations at specific sites, in either DNA or RNA. DNA base editors often comprise a fusion of a DNA-binding molecule that is fused to a catalytically active base-modification enzyme that acts on single-stranded DNAs. Said DNA binding molecule often is a catalytically inactive nuclease, such as a clustered regularly interspaced short palindromic repeat (CRISPR)-associated sequence (Cas) nuclease. So far, two types of DNA editors have been developed, a cytosine base editor (CBE) and an adenine base editor (ABE).

The term “guide ribonucleic acid (gRNA)”, as is used herein, refers to a single stranded RNA molecule, or to RNA molecules, that comprise a spacer sequence, also termed guide sequence and, optionally, a tracr sequence that serves as a binding scaffold for the Cas nuclease. As is known to a person skilled in the art, some Cas proteins such as Cpf1 do not require a separate tracr RNA. The term gRNA includes reference to “single guide RNA” (sgRNA) in case the guide and tracr are present on a single molecule, e.g. by linking the CRISPR RNA (crRNA), comprising the spacer sequence, and the tracr RNA via a synthetic loop. Said spacer sequence guides the Cas protein to a complementary strand of a target nucleic acid sequence. The sequence on the target nucleic acid that is complementary to the spacer sequence in the gRNA is termed protospacer. Said target nucleic acid may be single stranded or double stranded, RNA or DNA, depending on the type of CRISPR-Cas system. Said gRNA preferably has a length of at least 30 nucleotides, more preferred at least 34 nucleotides, more preferred at least 40 nucleotides, more preferred at least 46 nucleotides. Said gRNA preferably is less than 1000 nucleotides, preferably less than 200 nucleotides, preferably less than 100 nucleotides. Said gRNA molecule may include ribonucleic acid nucleotide analogues such as inosine, uridine, xanthine, hypoxanthine, 2,6-diaminopurine, and 6,8-diaminopurine-based ribonucleotides and deoxyribonucleotides. Cas protein(s) assembled around a gRNA guide will form a ribonucleoprotein (RNP) effector complex that specifically binds complementary target sequences.

The term “CRISPR-associated (Cas) protein”, as is used herein, refers to a protein that may be associated with a gRNA. CRISPR/Cas systems are presently grouped into two classes. Class 1 systems utilize multi-subunit Cas complexes, whereas Class 2 systems use only a single Cas protein to mediate its activity. Class 1, type III CRISPR-Cas systems have evolved to target especially RNA sequences. Unique proteins in these systems are Cas3 in Class 1 systems, Cas9 in Class 2 systems, and Cas10 in Class 1, type III systems.

The term “effector complex”, as is used herein, refers to a CRISPR-Cas ribonucleoprotein complex that binds a target nucleic acid sequence that comprises complementary sequences to the spacer sequence in the gRNA. Said complex comprises at least one gRNA and at least one Cas effector protein.

The term “deaminase”, as is used herein, refers to an enzyme that causes removal of an amine group from a compound. Said enzyme is often named according to the substrate, such as nucleotide. A nucleotide, as is known to a person skilled in the art, comprises a five-carbon sugar (2′-deoxyribose in DNA or ribose in RNA), a phosphate molecule, and a nitrogen-containing base selected from adenine, guanine, cytosine, thymine, and uracil. A deaminase often is named according to its substrate, e.g. an adenine deaminase, a cytidine deaminase, a guanidine deaminase, a thymidine deaminase, or an uracil deaminase.

The term “complementary strand”, as is used herein in the context of gRNA, refers to a target nucleic acid strand that is substantially complementary to, i.e. can base pair with, the spacer sequence of the gRNA. The complementary strand will base pair with, and bind to, the gRNA. Said complementary strand is either a target single stranded molecule, or one of the strands of a double stranded target molecule. In the latter case, binding of the gRNA to the complementary strand will displace the non-complementary strand of the target molecule.

The term “protospacer adjacent motif (PAM)”, as is used herein, refers to an additional nucleic acid sequence that is present on the target nucleic acid and resides adjacent to the protospacer sequence. The PAM size and location varies by CRISPR system and is typically a 2- to 5-bp sequence that may be located upstream or downstream of the protospacer motif. The presence of a PAM is required for unfolding of a double stranded target nucleic acid, such that the spacer sequence of a gRNA can find its complementary protospacer sequence on the target nucleic acid and base pair with it.

The term “cleavage-deficient”, as is used herein, refers to a Cas protein that is unable to cut the double stranded target nucleic acid. In the context of base-editing a double stranded target nucleic acid (dstna) molecule, the term refers to an enzyme that is not able to digest a dstna molecule, i.e. cut both strands of a dstna molecule. A cleavage deficient Cas may generate a nick in a dstna molecule, i.e. cut only one of the strands of a dstna molecule. A nickase-active variant can be generated, for example, by alanine substitution of an aspartate residue (D10A) of Cas9, producing a nick on the targeting strand, while the H840A alteration generates a nick on the non-targeting strand DNA (Jinek et al., 2012. Science 337: 816-821; Gasiunas et al., 2012. PNAS 109: E2579-E2586; Cong et al., 2013. Science 339: 819-823; Mali et al., 2013. Nature Biotech 31: 833-838). A R1138A mutant of LbCas12a and AsCas12a functions as a nickase ((Yamano et al., 2016. Cell 165: 949-62). Another existing nickase variant includes a FnCas12a K1013G/R1014G double mutant which can cut only the non-target strand (WO 2019/233990). Nicking of the non-edited strand may increase to efficiency of base editing. As is known to a person skilled in the art, a cleavage-deficient Cas protein may be a naturally occurring Cas protein, optionally together with an inhibitor that inhibits (ds) cleavage activity, or a mutated Cas protein. Examples of known cleavage-deficient mutants Cas proteins are dCas9(D10A and H840A) (Perez-Pinera et al., 2013. Nature Methods 10: 973-976), Cas9(D10A) and Cas9(H840A) (Shen et al., 2014. Nature Methods 11: 399-402), and nCas9 (Zong et al., 2018. Nature Biotech 36: 950-953). A relevant mutant of MmuC2C4 is D385A (mutation of aspartic acid at position 485 to alanine), which may result in an RNase deficient caspase. An AsCas12F1 cleavage deficient mutant comprises the amino acid alteration D225A.

The term “C2c4 nuclease”, as is used herein, refers to a class 2, Type V Cas protein that, belonging to the uncharacterized subtype V-U1 (Shmakov et al., 2017. Nature Rev Microbiol 15: 169-182, which is hereby incorporated by reference). A C2c4 nuclease is sometimes also referred to as Cas12u1. C2c4 nucleases have been described in Mycolicibacterium mucogenicum and other organisms including other Mycolicibacterium species such as M peregrinum and M. conceptionense, Mycobacterium species such as M. lentiflavum and M. colombiense, Clostridiales bacterium, Gordonia otitidis, Meiothermus silvanus, Pelobacter propionicus and Nocardia species such as N. pseudovaccinii. A C2c4 Cas protein binds double stranded DNA and, akin to most type V proteins, recognizes a 5′-TTN-3′ PAM on dstna.

The term “Cas12F nuclease”, as is used herein, refers to a class 2, Type V Cas12 protein that may cut either single or double stranded DNA. In general, Cas12 proteins are about 1,300 amino acids long. Cas12F proteins are on average about 500 amino acids long, and range from 422 amino acids to 603 amino acids (Karvelis et al., 2020. Nucleic Acids Res 48: 5016-5023). Cas12F family members have been isolated from a distinct organisms, including several unclassified archeaebacteria, Parageobacillus thermoglucosidasius, Acidibacillus sulfuroxidans, Ruminococcus spp., Syntrophomonas palmitatica, and Clostridium novyi (Karvelis et al., 2020. Nucleic Acids Res 48: 5016-5023, which is hereby incorporated by reference).

The term “inducible activity”, as is used herein, refers to an activity that is regulatable, preferably on a cell-by-cell basis, meaning that the activity can be induced in a specific cell or cell type. Said activity can be induced, for example, by exposing a cell to a compound, a temperature shift such as a cold or heat pulse, an acoustic signal and/or light, preferably with a wave length of more than 600 nm in the red or infrared spectrum. Preferred activities that are induced comprise activation of a DNA transcription factor, a DNA transcription co-factor, a DNA-modifying enzyme such as a DNA methyl transferase, or a DNA recognition site-specific recombinase.

The term “dimeric molecule”, as is used herein, refers to a molecule that comprises two subunits. A homodimeric molecule comprises two identical subunits, while a heterodimeric molecule comprises two different subunits. Said dimeric molecule may be a naturally occurring dimeric molecule, or a synthetic dimeric molecule which is formed, for example, by two parts of a naturally single molecule. A dimer protein may be formed without specific interaction domains, or by interaction of specific interaction domains, termed dimerization domains.

The term “chemically-sensitive dimerization domain”, as is used herein, refers to a dimerization domain that can be either induced or disrupted by a chemical compound. Said chemically-sensitive dimerization domain is, for example, based on dihydrofolate reductase (DHFR), DNA gyrase B (GyrB), and FK506 binding protein (FKBP) binding motifs that bind methotrexate, coumermycin, and rapamycin and FK506, respectively (Fegan et al., 2010. Chemical Reviews 110: 3315-36). A gibberellin-analog (GA3) has also been reported to act as a chemical dimerizer (Miyamoto et al., 2012. Nature Chem Biol 8: 465-70).

The term “light-sensitive dimerization domain”, as is used herein, refers to a dimerization domain that mediates light-inducible or light-disruptable protein-protein interaction domains. Said domains preferably require no exogenous ligands. However, systems that do need external cofactors, for example as described in Kyriakakis et al., 2018 (Kyriakakis et al., 2018. ACS Synth Biol 7: 706-717), are explicitly included herein. Preferred light-sensitive dimerization domains or optical dimerizer systems are, or are based on, the interacting domains of phytochromes and cryptochromes of bacteria and plants. Examples are known in the art and have been described, for example in Pathak et al, 2014. ACS Synth Biol 3: 832-838; Kennedy et al., 2010. Nature Methods 7: 973-975; and Taslimi et al., 2016. Nature Chem Biol 12: 425-430.

The term “linker sequence”, or linker, as is used herein, refers to a short amino acid sequence, preferably of 2-200 amino acids, that may be present between a cleavage-deficient Cas protein and a deaminase such as an adenine deaminase, a cytidine deaminase, a guanidine deaminase, a thymidine deaminase, or an uracil deaminase. Said linker may be used as a way of providing flexibility in order to modify or vary the editing window of the base editors. Such modifications will be apparent to a person of skill in the art and having reference to the accompanying examples. Said linker sequence may include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 amino acid residues.

The term “vector”, as is used herein, refers to a nucleic acid molecule capable of transporting genetic material to which it has been linked, or which is incorporated into the vector. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector is often used to transduce a gene encoding a protein of interest into a suitable host cell. Once in the host cell, the vector may replicate independently of, or coincidental with, the host chromosomal DNA. Examples of commonly used vectors are plasmids, viral vectors such as retroviral vectors, and bacteriophage-related vectors such as based on the E. coli M13 phage. A preferred viral vector is based on adeno associated virus (AAV), which allows small gene sizes to be inserted. In vitro, ex vivo and in vivo delivery of AAV vectors is described in Esvelt et al., 2013. Nat. Methods 10: 1116-1121; In vitro, ex vivo and in vivo delivery of lentiviral vectors is described in Shalem et al., 2014. Science 343: 84-87; In vitro, ex vivo and in vivo delivery of adenoviral vectors is described in Maddalo et al., 2014. Nature 516: 423-427).

The term “host cell”, as is used herein, includes a prokaryotic cell and a eukaryotic cell such as a yeast cell or a mammalian cell.

The term “prokaryote” as is used herein, refers to a cellular organism that lacks an envelope-enclosed nucleus. Cellular organisms with an nucleus enclosed within a nuclear envelope are referred to as eukaryotes. Prokaryotes include true bacteria (eubacteria), and archeae (archaeabacteria).

The term “eukaryotic cell”, as is used herein, refers to a fungal, plant, or animal, including human, cell.

The term “expression vector”, as is used herein, refers to a vector that is able to direct expression of one or more genes to which they are operatively-linked. Suitable regulatory elements include promoters, enhancers, internal ribosomal entry sites (IRES), and other expression control elements such as 5′ untranslated regions, optionally containing a ribosome binding site, 3′ untranslated region optionally comprising a ‘post stop-codon, ante terminator’ region, terminator sequences, and transcription termination signals such as polyadenylation signals and poly-U sequences. For more information the average skilled person is referred to, for example, in Goeddel, (1990), Gene Expression Technology in Methods in Enzymology vol 185, Academic Press. Regulatory elements include those giving direct constitutive expression in many types of host cell and those that direct expression of the nucleotide sequence only in certain cells (i.e., tissue-specific regulatory sequences). Regulatory elements may also direct expression in a temporal-dependent manner, such as in a cell-cycle dependent or developmental stage-dependent manner, which may or may not also be tissue or cell-type specific. Examples of promoters include pol I, pol II, pol III (e.g. U6 and H1 promoters). Examples of pol II promoters include, but are not limited to, retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the b-acting promoter, the phospho-glycerol kinase (PGK) promoter, and the EFla promoter. As well as promoters, regulatory elements may include enhancer elements, such as WPRE; CMV enhancers; the R-U5′ segment in LTR of HTLV-I; SV40 enhancer; and the intron sequence between exons 2 and 3 of rabbit b-globin. It will be appreciated by those skilled in the art that the design of the expression vector can depend on such factors as the choice of the host cell to be transformed, the level of expression desired, etc. Said regulatory elements such as promoter sequences may be an autologous sequences, or heterologous sequences, i.e. derived from a different species.

The term “tissue-specific promoter”, as is used herein, is a promoter that directs expression primarily in a desired tissue of interest, such as blood, specific organs (e.g., liver, pancreas), or particular cell types.

The term “plasmid”, as is used herein, refers to a circular double stranded DNA molecule into which additional DNA segments can be inserted, such as by using standard molecular cloning techniques. Plasmids often comprise an origin of replication and a marker gene that allows to identify a cell comprising the plasmid.

The term “viral vector”, as is used herein, includes reference to viral nucleic acid sequences that can be packaged into a viral particle. Examples of viral vectors include retroviruses, replication defective retroviruses, adenoviruses, replication defective adenoviruses, and adeno-associated viruses. Packaging is often mediated by a packaging signal on the viral nucleic acid molecule, and performed in an appropriate packaging cell that expresses the required proteins.

The terms “base pairing affinity” and “complementarity”, as are used herein, refer to the ability of a nucleic acid to form hydrogen bond(s) with another nucleic acid sequence by either traditional Watson-Crick base pairing or other non-traditional types. A percent identity (i.e. complementarity) in relation to a reference sequence, in the various descriptions of the invention, represents the percentage of residues in a nucleic acid molecule which can form hydrogen bonds (e.g., Watson-Crick base pairing) with a second nucleic acid sequence, preferably over the complete length of the shortest sequence (e.g., 5, 6, 7, 8, 9, 10 out of 10 being 50%, 60%, 70%, 80%, 90%, and 100% identity). “Perfectly complementary” means that all the contiguous residues of a nucleic acid sequence will hydrogen bond with the same number of contiguous residues in a second nucleic acid sequence. This is a preferred condition for antisense oligonucleotide bindinA to G targeting RNA which corresponds to 100% identity for a length of targeting RNA molecule which is the same length as the antisense oligonucleotide.

Also, the term “substantially complementary”, as is used herein, refers to a degree of identity that is at least 90%, 95%, 97%, 98%, 99%, or 100% between the portion of an antisense oligonucleotide and the equivalent length of a targeting RNA molecule. This may also correspond to nucleic acids that hybridize under stringent conditions.

The term “stringent conditions”, as used herein, in the context of hybridization conditions, refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, NY, 2001); and Tijssen, Laboratory Techniques in Biochemistry and Molecular Biology—Hybridization with Nucleic Acid Probes Part I, Chapter 2 (Elsevier, New York, 1993), each of which are incorporated herein by reference. The Tm is the temperature at which more than 50% of a given strand of a nucleic acid molecule is hybridized to its complementary strand.

### 4.2 Editing Methods

The methods of the invention allow A to G and C to T editing of the complementary strand of a target nucleic acid. For this, a gRNA directs a cleavage-deficient Cas nuclease that is fused to a adenine deaminase or a cytidine deaminase, to a double stranded target nucleic acid. The targeting gRNA molecule is designed to have complementarity with the target nucleic acid, where hybridization between a target sequence and the RNA targeting molecule promotes the formation of a RNA-targeting complex. Targeting RNA molecules in accordance with the invention are referred to herein as guide RNA (gRNA). In general, a targeting RNA has a sufficient complementarity with the target polynucleotide sequence to hybridize with the target sequence and direct sequence-specific binding of the CRISPR enzyme or Cascade complex to the target sequence. The degree of complementarity between a targeting RNA and its corresponding target sequence may be more than about 50%, 60%, 75%, 80%, 85%, 90%, 95%, 97.5%, 99%, or more, with optimal algorithmic alignment. Throughout this specification in any context, optimal alignment may be determined using, for example, any of the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (lllumina, San Diego, CA), SOAP (available at soap.genomics.org.cn), and Maq (available at aq.sourceforge.net). The alignment is preferably performed over the whole length of the spacer sequence.

A cell such as an isolated cell may be targeted with a CRISPR-based editing system according to the invention, comprising a gRNA and a cleavage-deficient Cas nuclease that is fused to a deaminase. According to the invention, double stranded (ds) DNA is targeted and modifications are introduced into the dsDNA sequence by enzymatically mediated chemical changes to the nucleotide residues. For base editing, all possible delivery modes, as also described herein, may be used. In addition, the cleavage-deficient Cas nuclease may be introduced into a cell separately, simultaneously or sequentially with an isolated gRNA. Moreover, the isolated gRNA and the cleavage-deficient Cas nuclease may be introduced into a cell by different means.

In certain uses of the products of the invention, individual components may be pre-assembled as a ribonucleoprotein (RNP) complex to achieve the desired target locus effects. Such RNP complexes may be introduced directly into cells for example by electroporation, by bombardment, using RNP-coated particles, by chemical transfection or by some other means of transport across a cell membrane.

For the in vitro assembly of a ribonucleoprotein complex, the required Cas protein or proteins are expressed and purified from a suitable expression system. Commonly used expression systems for heterologous protein production include E. coli, Bacillus spp., baculovirus, yeast, fungi, most preferably filamentous fungi or yeasts such as Saccharomyces cerevisiae and Pichia pastoris, eukaryotic cells such as Chinese Hamster Ovary cells (CHO), human embryonic kidney (HEK) cells and PER.C6® cells (Thermo Fisher Scientific, MA, USA), and plants. The efficiency of expression of recombinant proteins in heterologous systems depends on many factors, both on the transcriptional level and the translational level.

Cas proteins preferably are produced using prokaryotic cells, preferably E. coli. Said Cas proteins are preferably produced by expression cloning of the proteins in a prokaryotic cell of interest, preferably E. coli. For this, an expression construct, preferably DNA, is preferably produced by recombinant technologies, including the use of polymerases, restriction enzymes, and ligases, as is known to a skilled person. Alternatively, said expression construct is provided by artificial gene synthesis, for example by synthesis of partially or completely overlapping oligonucleotides, or by a combination of organic chemistry and recombinant technologies, as is known to the skilled person.

As an alternative, or in addition, Cas proteins may be isolated from a thermophilic organism by expression of a tagged Cas protein or proteins in said thermophilic organism, and isolation of ribonucleoprotein complex comprising said Cas proteins on the basis of the tag. Said isolated ribonucleoprotein complexes can be isolated using the tagged Cas protein.

Said expression construct is preferably codon-optimised to enhance expression of the Cas proteins in a prokaryotic cell of interest, preferably E. coli. Further optimization may include the removal of cryptic splice sites, removal of cryptic polyA tails and/or removal of sequences that may lead to unfavorable folding of the mRNA. In addition, the expression construct may encode a protein export signal for secretion of the Cas proteins out of the cell into the periplasm of prokaryotes, allowing efficient purification of the Cas proteins.

Methods for purification of Cas proteins are known in the art and are generally based on chromatography such as affinity chromatography and ion exchange chromatography, to remove contaminants. In addition to contaminants, it may also be necessary to remove undesirable derivatives of the product itself such as degradation products and aggregates. Suitable purification process steps are provided in Berthold and Walter, 1994 (Berthold and Walter, 1994. Biologicals 22: 135-150).

As an alternative, or in addition, a recombinant Cas protein or proteins may be tagged with one or more specific tags by genetic engineering to allow attachment of the protein to a column that is specific to the tag and therefore be isolated from impurities. The purified protein is then exchanged from the affinity column with a decoupling reagent. The method has been routinely applied for purifying recombinant protein. Conventional tags for proteins, such as histidine tag, are used with an affinity column that specifically captures the tag (e.g., a Ni-IDA column for the histidine tag) to isolate the protein from other impurities. The protein is then exchanged from the column using a decoupling reagent according to the specific tag (e.g., imidazole for histidine tag). This method is more specific, when compared with traditional purification methods.

Suitable tags include c-myc domain (EQKLISEEDL), hemagglutinin tag (YPYDVPDYA), maltose-binding protein, glutathione-S-transferase, FLAG tag peptide, biotin acceptor peptide, streptavidin-binding peptide and calmodulin-binding peptide, as presented in Chatterjee, 2006 (Chatterjee, 2006. Cur Opin Biotech 17, 353-358). Methods for employing these tags are known in the art and may be used for purifying a Cas protein or proteins.

Methods for expression proteins in E. coli are known in the art and can be used for expression and purification of the Cas-proteins.

In a preferred method, Cas proteins are expressed in E. coli from a codon-optimized expression construct. Said construct is placed in a bicistronic expression plasmid containing a Strep-tag and amino-acid sequence Glu-Asn-Leu-Tyr-Phe-Gln-(Gly/Ser) at the N-terminus, which amino acid sequence is recognized by a Tobacco Etch Virus (TEV) protease. The expression plasmid is transformed into E. coli, for example in strain B121(DE3). Following growth at 37° C. in a desired culture volume, until OD600 of ˜0.6, the culture is placed on ice for 1 hour after which isopropyl 8-D-1-thiogalactopyranoside is added to a final concentration of 0.1 mM. The culture is then incubated at 18° C. for ˜16 hours (overnight). The cells are harvested and lysed in Buffer A (100 mM Tris-HCl, 150 mM NaCl) by sonication and subsequently spun down at 30.000 g for 45 min. The clarified lysate in filtered and run over a pre-equilibrated StrepTrap FPLC column (GE Healthcare, Chicago, IL). After washing the column with Buffer A until no more protein is present in the flow through, the protein of interest is eluted using Buffer B (100 mM Tris-HCl, 150 mM NaCl & 2.5 mM D-desthiobiotin). The protein is cleaved from the affinity tag by addition of TEV protease and left to incubate overnight at 4° C. The protein of interest is separated from the mixture by a HisTrap and StrepTrap affinity chromatography step, from which the flow through is collected. If required, an additional size exclusion chromatography may be added to achieve higher purity.

In certain uses of the products of the invention, individual components may be expressed in a cell to form a ribonucleoprotein (RNP) complex to achieve the desired target locus effects. Such individual components may be expressed from suitable nucleic acid expression constructs that are introduced into a cell. Said introduction of suitable expression constructs into a cell may be vector mediated, or non-vector mediated.

Methods of non-vector-mediated delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™, and SAINT™). Cationic and neutral lipids that are suitable for efficient receptor-recognition lipofection of polynucleotides include those of WO 91/17424 and WO 91/16024. Delivery can be to cells (e.g. in vitro or ex vivo administration) or target tissues (e.g. in vivo administration.

Methods of vector-mediated delivery include infection and transfection by employing, for example, a vector such as an expression vector.

Base editing in the context of the present invention involves site-specific modification of the DNA base, preferably along with manipulation of the DNA repair machinery to avoid faithful repair of the modified base. The base editors of the invention are chimeric proteins composed of a cleavage-deficient Cas nuclease, for example a C2C4 or Cas12F Cas nuclease, together with targeting RNA to form an RNP complex, and a catalytic domain capable of deaminating a cytidine or adenine base. Advantageously, usage of a cleavage-deficient Cas nuclease, such as a MmuCas12u1, there is no generating of double strand breaks giving rise to insertions and deletions (indels) at target and off-target sites.

Said catalytic domain capable of deaminating a cytidine or adenine base preferably is connected to the cleavage-deficient Cas nuclease through a linker of 1-200 amino acids residues, preferably a linker of 5-50 amino acid residues. The presence of a linker is taught to provide some flexibility to the catalytic domain, allowing it to deaminate cytidine or adenine base residues on the non-target strand, and within a region of 1-25 nucleotides adjacent to a PAM sequence on the target strand. As is known to a person skilled in the art, said PAM sequence is 5′ TTN for MmuBE_E1, 5′-NGG for Cas9, 5′-(T)TTV for Cas 12a, and 5′-TTTR for Cas12f, whereby R is G or A.

G to A editing within the non-target strand, resulting from C to T editing of the target strand, was observed over a stretch of nucleotides downstream of a PAM sequence. Base editing may be concentrated in one of more regions adjacent to a PAM sequence, for example in a PAM-proximal region and a PAM-distal region. Said PAM-proximal region is a region close to the PAM sequence, for example between 1-10 nucleotides from the PAM sequence, such as 3-7 nucleotides from the PAM sequence. Said PAM-distal region is a region at some distance from the PAM sequence, for example between 10-25 nucleotides from the PAM sequence, such as 14-20 nucleotides, 15-19 nucleotides, or 16-18 nucleotides.

Hydrolytic deamination of adenine (A) and cytidine (C) into inosine (I) and uridine (U) means these will be read as guanosine (G) and thymine (T), respectively, by polymerase enzymes. The conversion of C into U might result in the onset of base excision repair, where a U from the DNA is excised by uracil DNA N-glycosylase. This is followed by a repair into C through error-free repair or error-prone repair that results in base substitutions. Blocking the base excision is promoted by the use of uracil DNA glycosylase inhibitor (UGI).

Cytidine deaminase-based DNA base editors catalyze the conversion of cytosine into uracil, for example APOBEC deaminase such as APOBEC3 cytidine deaminase, which converts cytidine into thymidine. In the base-editing system, APOBEC, guided by a cleavage-deficient Cas nuclease, deaminates a specific cytidine to uracil; the resulting U-G mismatches are resolved via repair mechanisms and form U-A base pairs, and subsequently T-A base pairs. Thus, these base editors can be used to produce C-to-T point mutations (in dsDNA: C:G to T:A).

Cytidine deaminase converts C into U and subsequently uracil DNA glycosylase can perform error-free repair, converting the U into the wild-type sequence. The addition of the UGI inhibits the base excision repair pathway, resulting in a three-fold increased efficiency.

Multiple additional base-editing systems can be made in accordance with the invention, with different deaminases and/or with different further domains. For example, an activation-induced cytidine deaminase domain (MD) may be linked to the Cas-base edit, optionally with an UGI domain. Because the activity of the UGI inhibits excision repair and improves the base-editing efficiency, two UGI domains can be included; e.g. one at the C- and one at the N-terminus of the cleavage-deficient Cas nuclease.

In terms of what determines the best base editor for a given application, the choice of base editor will depend on the availability of a PAM sequence, the presence of a C nucleotide relative to the PAM, and how the base-editor reagents are delivered to the target cell. Furthermore, the nature of the edits could also be determined by the base editor.

Adenine base editors may be made in accordance with the invention to modify adenine bases. The deamination of adenine yields inosine, which can base pair with cytidine and subsequently be corrected to guanine, thereby converting A into G, or A-T into G-C.

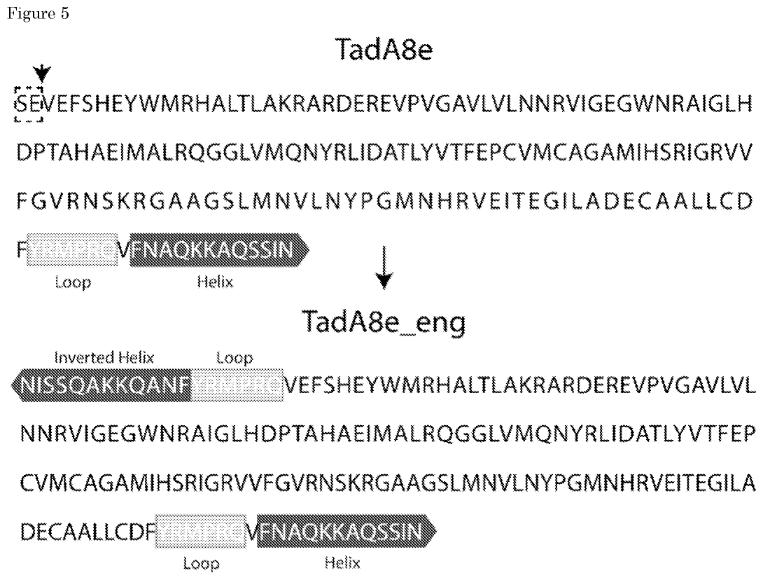

Said adenine base editor may be altered. For example, said adenine base editors may contain mutations that increase activity. Said mutations may offer improved editing efficiencies and induce processiveness of the adenine base editor. For example, said adenine base editor includes ABE8e, as described in Richter et al., 2020 (Richter et al., 2020. Nature Biotech 38: 883-891), which is herein incorporated by reference. Said adenine base editor may include further altered deaminase domains, for example a deaminase comprising a duplicated C-terminal region at the N-terminus. Said duplicated region may comprise loop-helix-domains, such as present at the C-terminus of ABE8e. Said duplicated region, or part of it, may be inverted at the N-terminus. For example, an altered deaminase may comprise a C-terminal loop domain in inverted orientation duplicated at the N-terminus, for example the amino acid sequence FNAQKKAQSSIN may be positioned as NISSQAKKQANF at the N-terminus of a deaminase, followed by a YRMPRQ loop region. A preferred deaminase comprises the amino acid sequence NISSQAKKQANFYRMPRQ at the N-terminus, whereby optionally the amino acids SE have been removed from the N-terminus. A sequence of an engineered A to G deaminase is provided in FIG. 5, termed TdA8e_eng.

To accomplish A to G editing of the complementary strand, said nuclease deficient Cas is of a limited size. Said nuclease deficient Cas may lack at least part of a recognition lobe, termed Rec1 and Rec2 domains, when compared to other Cas nucleases such as Cas9, Cas12a and Cas12b nucleases. Said nuclease deficient Cas may lack most of a recognition lobe, such as a complete recognition lobe.

As an alternative, or in addition, said Cas nuclease preferably comprises 300-700 amino acid residues, preferably less than 650 amino acid residues, less than 600 amino acid residues, less than 550 amino acid residues, less than 500 amino acid residues, less than 450 amino acid residues, less than 400 amino acid residues. A cleavage-deficient Cas nuclease may comprise 695 or 422 amino acid residues.

Said nuclease deficient Cas may be selected from naturally occurring Cas nuclease, such as a c2c4 nuclease, a Cas phi or Cas12j (Pausch et al., 2020. Science 369: 333-337), CyaCas12u2, isolated from Cyanothece sp., LaeCas12u3, isolated from Lyngbya aestuarii, NaICas12u4, isolated from Nocardiopsis alba, RmuCas12u4, isolated from Rothia mucilaginosa, and Cas12F nuclease. A c2c4 nuclease such as Mmuc2c4 was found to be a natural nuclease deficient Cas.

In summary, base editors using cytosine deaminases can convert C-G via U-G into T-A, and adenine deaminases can convert A-T via I-C into G-C. These base modifications can generate targeted sequence variation in a precise manner. The cellular repair machinery will repair the non-edited strand using information from the complementary edited template. The nuclease deficient Cas protein as described herein, when fused to a adenine deaminase, preferably at the C-terminus of the Cas protein, allows A to G editing of the complementary strand of the target DNA.

Methods of the invention may be in vitro, for example they are performed using a synthetic mix of the reaction components in a suitable buffer system. In some in vitro embodiments there is used a cell-free transcription/translation system.

Methods of the invention may be employed occurring ex vivo, for example in a cell or cell culture. In ex vivo treatments, diseased cells are removed from the body, treated with a base editor of the invention, and then transplanted back into the patient. Ex vivo editing has an advantage of allowing the target cell population to be well defined and the specific dosage of therapeutic molecules delivered to cells to be specified. In one aspect, the invention provides therapeutic methods for organisms (humans or animals), whereby a single cell or a population of cells is sampled or cultured. Said cell or cells may then be modified ex vivo, as described herein, and then re-introduced into the organism. The cells modified ex vivo may be stem cells, whether embryonic or induce pluripotent or totipotent stem cells, including totipotent stem cells, which may preferably be non-human totipotent stem cells.

In vivo embodiments are also provided. In vivo editing can be used advantageously from this disclosure and the knowledge in the art.

### EXAMPLES

### Example 1

### Materials and Methods

S. cerevisiae Plasmid Construction

The plasmids constructed in this study and the oligonucleotides (IDT) used for cloning and sequencing can be found in Tables 1 and 2, respectively. Mmu base editors in S. cerevisiae were genome integrated to generate various strains expressing different targeting guides expressed from a multicopy plasmid (Table 3). CRISPR arrays for Cas12a and MmuCas12u1 were expressed under control of the SNR52 promoter on a PL-074 backbone.

Initially, PL-074 was constructed to correct the SUP4 terminator sequence to its original length, by PCR amplification of pUD628 and subsequently re-circularizing it by blunt-end ligation. PL-098 was constructed by incorporation of the INT1 spacer (Verwaal et al., 2018. Yeast 35: 201-211) as an overhang in the forward primer used for linearization of PL-074 by PCR amplification. In order to incorporate the MmuCas12u1 repeats, PL-162 was built by restriction digestion of pCRISPR_NT (BbsI), containing a spacer flanked by BbsI sites, with BbsI-HF® and ligation with a spacer created by annealing oligonucleotides BG19061 and BG19062. PL-162 was then used to amplify the MmuCas12u1 CRISPR array containing a spacer flanked by BsaXI restriction sites instead of BbsI (fragment A0185). A0185 was digested in a two-step protocol with restriction enzyme KpnI and BtgZI. Afterwards, staggered ends were removed by T4 DNA polymerase (NEB). The blunted product was ligated into PCR amplified PL-074, to construct PL-163. PL-139 was constructed using the same protocol, except that a non-targeting spacer fragment obtained by annealing two oligonucleotides was used instead for ligation to BbsI restriction digested pCRISPR_NT (BbsI), obtaining the intermediate plasmid PL-138.

For easy screening correctly assembled plasmids, PL-196 was built which contains a rfp gene between the MmuCas12u1 repeats. PL-196 was constructed by HiFi® assembly of four PCR amplified fragments. Two backbone fragments were obtained from PL-163 and two RFP expression cassette fragments were obtained from pCRISPR-Cas12a-entry. Subsequently, MmuCas12u1 CRISPR array plasmids were built by BsaXI digestion of PL-196 and ligation of annealed oligonucleotide pairs with adequate overhangs.

### Fluorescence Repression Assay

The pTarget-GFP plasmid was constructed using BamHI restriction and ligation of a linear P_{lacIq} and GFP gene fragment amplified from the pTarget-PS plasmid, comprising the PAM-SCANR NOT gate-based circuit in a pAU66 plasmid backbone. pTarget-GFP containing different PAMs were constructed by site directed mutagenesis. The pTarget-operon plasmid was constructed by digesting the pTarget-GFP plasmid with BamHI enzyme to generate a linear vector which was assembled with an mRFP fragment containing compatible overhangs using the NEBuilder® HiFi DNA Assembly. The pTarget-divergent plasmid was constructed using a fragment of pTarget-GFP digested with the restriction enzymes, AatII and BamHI and subsequent ligated with a mRFP fragment under the control of a Taq promoter.

For the GFP silencing assays, E. coli cells harbouring pTarget-GFP and pCRISPR-GFP were made chemically competent and transformed with different Mmu base editor (pCas) plasmids, as indicated. After recovery, the transformation mix was diluted 2 μL:200 μL M9TG medium in a 96 well 2 mL master block (Greiner). Master block was then sealed using a gas-permeable membrane (Sigma, AeraSeal™) and grown overnight at 37° C. at 900 rpm overnight. The following day, the cells were diluted 1:10000 in fresh M9TG medium in a 96-wells master block and grown overnight at 37° C. Overnight cultures were then used for fluorescence measurements.

### Plate Reader Measurements

Overnight cultures were diluted 1:10 in 200 ˜L PBS and measured on a Biotek Synergy MX microplate reader a Synergy MX microplate reader. Cell density was measured with 600 nm and GFP fluorescence was measured with an excitation of 405 nm and emission of 508 nm. GFP was measured using a gain of 50, 75 and 100. Fluorescence was calculated as:

$$\frac{average ( \frac{{Fl}_{{x}_{targeting}} - {Fl}_{Blank}}{OD {600}_{{x}_{targeting}} - OD {600}_{Blank}} )}{average ( \frac{{Fl}_{FS} - {Fl}_{Blank}}{OD {600}_{FS} - OD {600}_{Blank}} )}$$

### Base Editing Assay

E. coli cells harboring pCRISPR-C-tile or pCRISPR-C motif plasmids and their corresponding pTarget plasmids were made chemically competent and transformed with the different Mmu base editor (pCas) plasmids. After recovery, the transformation mix was diluted 2 μL:200 ∥L M9TG medium in a 96 well 2 mL master block (Greiner). Master block was then sealed using a gas-permeable membrane (Sigma, AeraSeal™) and grown overnight at 37° C. at 900 rpm overnight. The following day, the cells were diluted 1:10000 in fresh M9TG medium in a 96-wells master block and grown overnight at 37° C. 20 μL E. coli cultures were taken every at time point 16, 24 and 48 hours for C-tile base editing, whereas samples were only taken at 40 hours for C-motif base editing. Base edited region was PCR amplified by using 2 μL cultures in a 50 μL PCR reaction using Q5® High-Fidelity 2X Master Mix (NEB). Amplified fragments were purified using DNA Clean & Concentrator™5 (Zymo Research) and sequenced.

S. cerevisiae Transformations

In order to construct a S. cerevisiae strain with genomic integration of egfp, an egfp expression cassette was integrated into integration site 1 (INT1) (Verwaal et al., 2018. Yeast 35: 201-211). A S. cerevisiae strain harboring pUDE731 (YSTB013) was transformed with 500 ng of PL-098 and four linear DNA fragments by the LiAc/SS carrier DNA/PEG method (Gietz and Woods, 2002. In “Methods in enzymology”, Elsevier 350: 87-96): one containing the Kluyveromyces lactis promoter of KLLA0F20031g (kl11p); another harboring the egfp gene and the CYCc terminator from pCFB2791 and two linear fragments homologous to the INT1 site as previously described (281). Correctly assembled and integrated cells were assessed by colony PCR and sequencing with primers listed in Table 1. After sequential sub-culturing in liquid YPD and a last culture on YPD-agar for plasmid curing. One colony isolate was selected and named YSTB164.

Subsequently, strains YSTB305 and YSTB211 were transformed with plasmids PL-242 to PL-246 and PL-139. Obtained colonies were investigated for phenotype change (red pigment accumulation in case of ade2 knockouts).

Base Editing Assessment in S. cerevisiae

Red colonies were picked and re-streaked on YPD+G418 media until single red colonies were isolated. Individual colonies were picked for genomic DNA amplification using Q5® High-Fidelity 2X Master Mix (NEB). PCR products were analyzed by Sanger sequencing (Macrogen).

### Results

### Smallest C to T Base Editor Edits in Two Regions

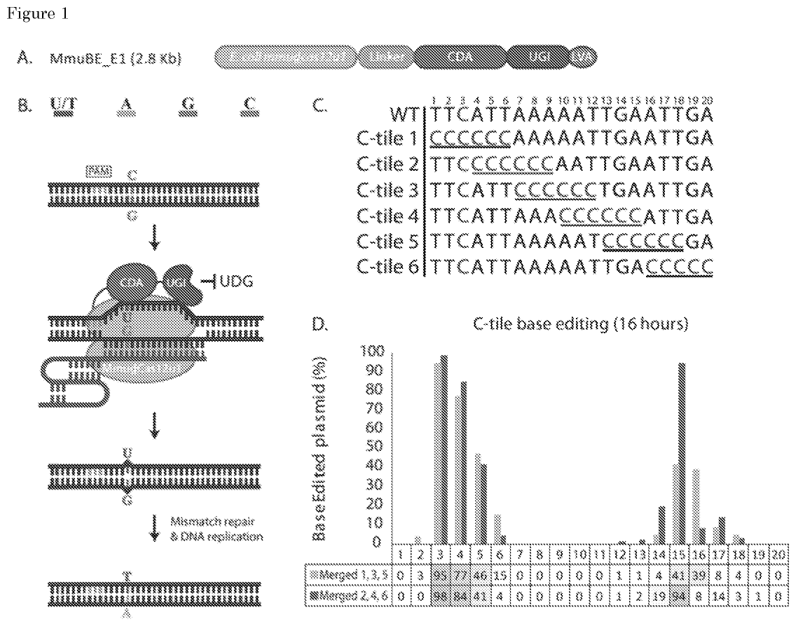

The first MmuCas12u1 base editor (MmuBE) constructed in this work consists of a catalytically inactive MmuCas12u1, termed dead MmuCas12u1 (MmudCas12u1), a 121-amino acid linker, a cytidine deaminase protein CDA, an uracil glycosylase inhibitor UGI, and an LVA degradation tag to reduce toxicity of the base editor (FIG. 1A) (Banno et al., 2018. Nature Microbiol 3: 423-429). This first construct is termed MmuBE_E1, based on the nomenclature of the prokaryotic Cas9 base editors (Banno et al., 2018. Nature Microbiol 3: 423-429). A test for base editing was developed, by growing E. coli cells harboring 3 plasmids: pCas, pCRISPR and pTarget. pCas and pCRISPR express the base editor and the CRISPR array, respectively, whereas pTarget plasmids contain the protospacer target sequence (FIG. 1B). We generated six variants of pTarget, which had six consecutive Cytosine bases at six different positions of the protospacers, termed C-tile plasmids (FIG. 1C). These boxes of six C's shift 3 positions towards the 3′ end until the 20th position is reached (FIG. 1C). This method ensures overall C coverage on the protospacer. In addition to the C-tiles, a C at position 3 (C3) was also always present and served as an internal standard for base editing. E. coli cells harboring all three plasmids were grown for 48 hours. Samples were taken at time points 16, 24 and 48 hours and were used for PCR amplification. Subsequent deep sequencing of the obtained amplicons was performed to assess base editing of the whole population. Sequence analysis revealed that base editing occurred in each of the six C-tile plasmids, with efficient C3 base editing (>90% editing) in all plasmids (data not shown). Next, the results of the uneven C-tile plasmids (1, 3 and 5) and of the even C-tile plasmids (2, 4 and 6) were merged to reveal the base editing window (FIG. 1D, and data not shown). Interestingly, it was found that MmuBE_E1 catalyzes base editing in two different regions within the base editing window instead of the one found for previously described base editors (Rees and Liu, 2018. Nature Reviews Genet 19: 770-788). These regions consist of a PAM-proximal (positions 2-5) and PAM-distal (positions 13-19) region. Positions 3 and 4 were found to have the highest base editing efficiency of >75%. Base editing efficiency for C15 varied between plasmids (C-tile 5 and C-tile 6). C15 base editing was found to be 41% for C-tile 5 and 94% for C-tile 6. These differences are most pronounced in position C15 but can also be seen for other positions, such as C6, C16 and C17. This may be caused by sequence specific base editing biases i.e., context dependent base editing. In an attempt to reduce the second base editing region, the spacer length was reduced, ranging from 14-17 nt (data not shown). The use of shorter spacers leads to shorter R-loops, which reduces the availability of ssDNA on the 3′ of the protospacer and thereby the base editing in that region (data not shown). From Sanger sequencing data of the whole population, a spacer length of 14 nt was found to be able to reduce the extension of the 2nd base window to positions 14-16 (data not shown). However, this approach also increased the likelihood of off-targeting. For that reason, a different approach was taken to reduce the base editing window, as described below.

Characterization of Various MmuBEs in E. coli

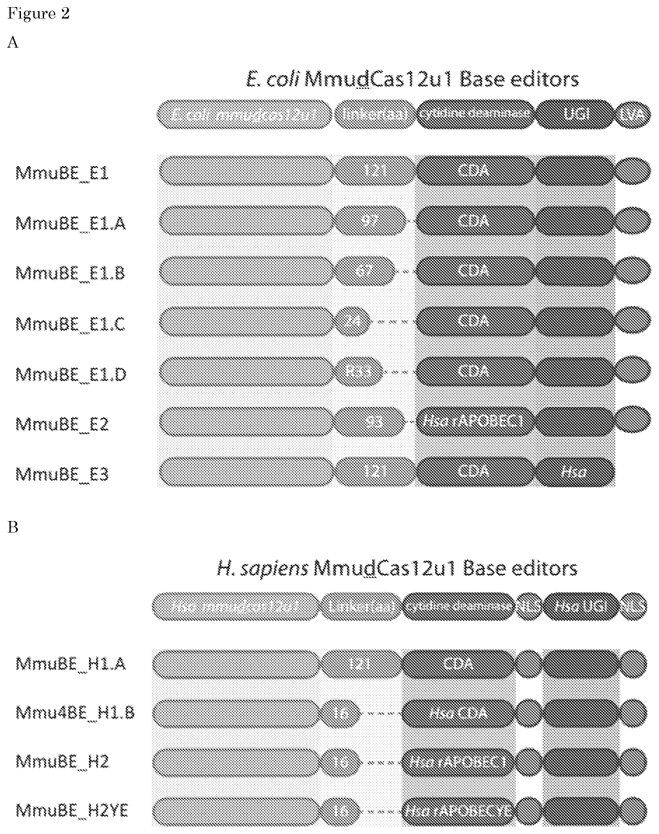

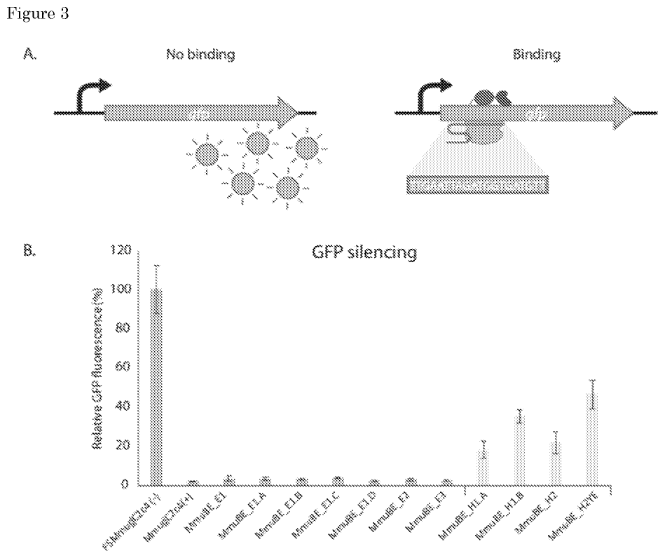

Various MmuBEs were designed by varying the deaminase module as well as the linker length (FIG. 1). Linker variation consisted of trimming down the flexible linker that was used in MmuBE_E1 from 121 to 97, 67 and 29 amino acids (aa). In addition, a rigid linker (33 aa) was tested as well (Tan et al., 2019. Nature Commun 10: 1-10). MmuBE_E1 base editors with these linkers were named MmuBE_E1.A-D (FIG. 2). Next to creating E. coli MmuBEs, several MmuBEs were also constructed for editing of mammalian cells. For constructing these MmuBE_H variants, we used H. sapiens codon harmonized mmudcas12u1, H. sapiens optimized cytidine deaminases (CDA or rAPOBEC1) and H. sapiens optimized uracil glycosylase inhibitor (UGI). The MmuBE_H1.A and MmuBE_H1. B variants contain CDA and UGI fused with a 121 aa or 16 aa linker, respectively. Using the same 16 aa linker, MmuBE_H2 and MmuBE_H2YE were constructed using rAPOBEC1 and rAPOBEC1 YE, respectively (Komor et al., 2016. Nature 533: 420-424). rAPOBECLYE was previously shown to have a narrower editing window compared to WT rAPOBEC1 (Kim et al., 2017. Nature Biotech 35: 371-376; Li et al., 2018. Nature Biotech 36: 324-327). MmuBE_H variants were also tested in E. coli to validate their base editing potential, prior to testing in human cells.

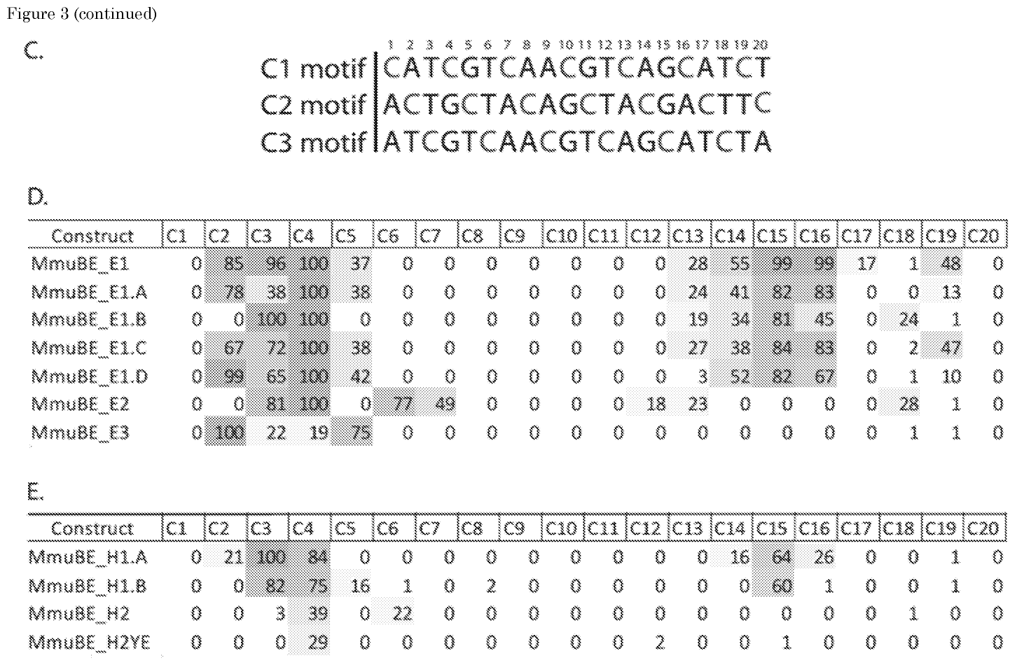

Prior to base editing, all MmuBEs were tested for binding activity of MmudCas12u1 in vivo using a GFP silencing assay. MmuBEs targeted a short GFP sequence containing no C nucleotide (only A, G or T nucleotides), so C-to-T base editing of the target sequence cannot occur (FIG. 3A). A frame shift E. coli MmudCas12u1 (FSdMmu) and E. coli MmudCas12u1 were included to function as negative and positive controls, respectively. GFP fluorescence was measured and normalized to FSdMmu (FIG. 3B) and therefore, all percentages showed in FIG. 3B are relative to the fluorescence of this strain. It was found that all E. coli MmuBEs (MmuBE_E) were able to bind to the target DNA, i.e. decreasing the GFP levels to <5% when compared to the negative control levels. MmuBE_E base editors silenced GFP similarly to the positive control dMmu (E. coli harmonized). As for MmuBE_H base editors, all MmuBE_H were found to have lower silencing activity when compared to the dMmu control, with 20-50% of GFP fluorescence still being detected. Out of the MmuBE_H base editors, MmuBE_H1.A and MmuBE_H2 show the best silencing activity with only 18% and 22% GFP fluorescence detected, respectively. This is followed by MmuBE_H1.B with 35% GFP fluorescence and then MmuBE_H2YE with the least silencing, with 47% of GFP fluorescence still being detected. Difference in silencing between MmuBE_E and MmuBE_H base editors can be due to expression differences affected by codon usage of E. coli. After testing the binding activity of various MmuBEs, base editing activity was tested.

The different C motif plasmids contain a tiled C motif (CxxCxxCxxCxxCxxCxxC), starting at every first (C1 motif), second (C2 motif) or third (C3 motif) nucleotide of the protospacer (FIG. 3C). Cells containing pCas (expressing Mmu BE), pCRISPR (expressing CRISPR array) and C-motif plasmids were grown for 48 hours and were used for a population PCR, which amplified the protospacer region on the C-motif plasmids. Amplified products were sequenced and results were analyzed by EditR (Kluesner et al., 2018. CRISPR J 1: 239-250). Base editing results obtained from all three C motif plasmids were merged and visualized in a heatmap (FIG. 3D). It was found that trimming the MmuBE_E1 linker from 121 aa to 24 aa (MmuBE_E1.C) had no effect on editing of either of the two base editing regions (FIG. 3D). However, MmuBE_E1.D, containing a 33 aa rigid linker showed slightly lower base editing activity in the PAM-distal region. Unexpectedly, also MmuBE_E2 and MmuBE_E3, which have long flexible linkers (93 aa and 121 aa), showed reduction of the PAM-distal region. MmuBE_E2 contains a H. sapiens optimized rAPOBEC1 instead of CDA and MmuBE_E3 contains a H. sapiens optimized UGI instead of the E. coli optimized UGI. Expression of these H. sapiens optimized genes in E. coli probably affect folding of the fusion proteins thereby changing the total number of active Mmu_BE proteins in the cell. Next, MmuBE_H base editors were also found be active in E. coli, although they show lower base editing activity compared to MmuBE_E base editors (FIG. 3E). MmuBE_H1.A and MmuBE_H1.B also have two base editing regions, but with reduced overall activities. MmuBE_H1.A edits C's at position 2-4 and 14-16, whereas MmuBE_HLB (containing a shorter linker of 16 aa) edits C′s at position 3-6 and 15-16. This suggests that, in these constructs, linker reduction from 93 to 16 aa results in a slight shift of the PAM-proximal base editing region.

The most precise MmuBEs in E. coli were found to be MmuBE_H2 and MmuBE_H2YE, with base editing detected only in the PAM-proximal region (FIG. 3E). MmuBE_H2 edits C's at position 3, 5 and 6, whereas MmuBE_H2YE only edits at position 4 with little to no editing found at position 12 and 15. However, although MmuBE_H2 and MmuBE_H2YE have a narrow editing range, it should be mentioned that both base editors have a significantly lower base editing activity when compared to other MmuBEs. Hence, the detected narrow base editing window appears to be a consequence of a lower editing efficiency. The reduced editing activity may have different explanations: increased expression of human-codon optimized mmudcas12u1 (in line with aforementioned reduction of silencing efficiency), of Hsa-APOBEC1-type cytosine deaminase, and of human-codon optimized uracil glycosylase inhibitor (Hsa-UGI). All these MmuBEs should still be analyzed by deep sequencing to validate the presented results obtained by Sanger Sequencing. Nonetheless, a variety of MmuBEs was created with differences in base editing windows, providing a wide selection of MmuBEs and further expanding the base editing toolbox in E. coli.

MmuBE Base Edits in S. cerevisiae

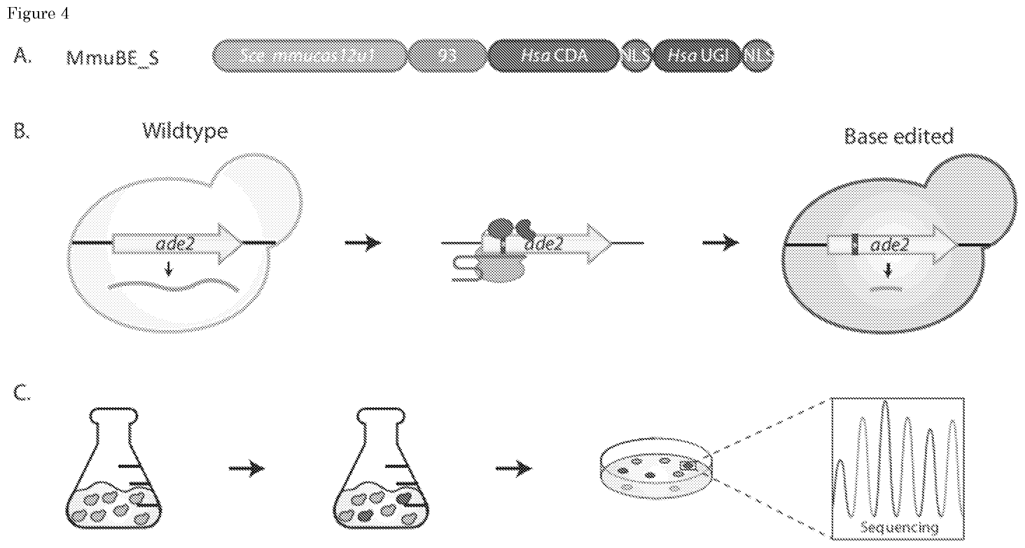

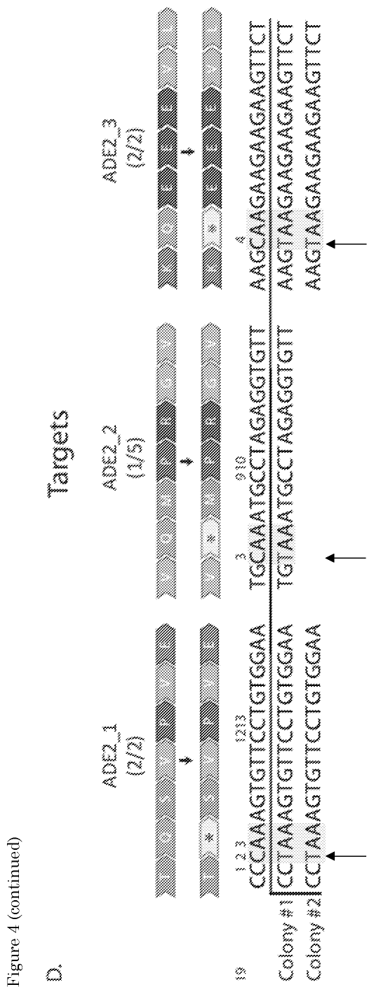

To check whether a MmuBE can also function in eukaryotes, a MmuBE_S was constructed and tested in Saccharomyces cerevisiae. MmuBE_S, contains a S. cerevisiae codon-optimized mmucas12u1, a 93aa linker, and human codon-optimized variants of CDA and UGI (FIG. 4A). Apart from the S. cerevisiae optimized mmucas12u1, MmuBE_S is similar to MmuBE_H1.A. Mmu_BE_S targeted the ade2 reporter gene in the genome of S. cerevisiae. Targeted C to T mutation in certain positions in ade2 results in the introduction of a premature stop codon, disrupting the ade2 gene. In the absence of adenine and when ade2 is knocked out, S. cerevisiae accumulates an intermediate of the adenine biosynthetic pathway (P-ribosylamino imidazole), which in aerobic conditions is oxidized to become a red pigment that can be visualized as red colonies on plates, easily discriminated from the white wild type (ade2+) colonies (FIG. 4B). Red colonies were selected for colony PCR and subsequent analysis of the obtained amplicons was performed by Sanger sequencing to confirm targeted base editing of the ade2 gene (FIG. 4C). By varying the crRNA guides, MmuBE_S targeted three position in the ade2 gene, of which C to T mutation in position 2, 3, or 4, respectively, leads to a nonsense mutation by converting a glutamine (Q) codon (CAA) to a stop codon (TAA) (FIG. 4C). Selected colonies were sent for sequencing of the three different targets, ADE2_1, ADE2_2 and ADE2_3. The sequencing results of the three targets, revealed that two out of two (2/2), one out of five (1/5) and two out two (2/2) were found to have the designed C to T base editing, respectively (FIG. 4C). Some red colonies did not contain targeted C to T mutations, such as the ones found in ADE2_2 and non-targeting samples. These clones appeared to be ade2 frame shift mutants, either due to spontaneous deletions or insertions. In addition, some red colonies were also found to have off-target base editing in the ADE2 gene, causing missense mutations, P508L and P472L (data not shown). Based on these initial analyses demonstrate that targeted Mmu-dependent base editing in S. cerevisiae is possible.

TABLE 1
Oligonucleotides used.

| | | |
|---|---|---|
| oligo ID | sequence (5′-3′) | description |

Construction of RFP-UGI entry plasmid

| | | |
|---|---|---|
| [symbol] | GATGTCCTCCTGAGCTCGC | [symbol] |
| [symbol] | AAGCTTGGCTGTTTTGGCG | [symbol] |
| [symbol] | ACGAGCTGTACAAGACTAGTCCCAAGAAGAAACGGAAAGT | [symbol] |
| [symbol] | CGCCAAAACAGCCAAGCTTTTAGACTTCCTCTTCTTCTTG | [symbol] |
| [symbol] | GCGAGCTCAGGAGGACCATATGGTGTCTAAGGGCGAAGAG | [symbol] |
| [symbol] | ACTAGTCTTGTACAGCTCGTCCATGC | [symbol] |
| [symbol] | CCCAAGAAGAAACGGAAAGT | [symbol] |
| [symbol] | CACTTTCCGTTTCTTCCTTGGCAAGTCTTCGTTAAGCACCGGTGGAGTG | [symbol] |
| [symbol] | GCGAGCTCAGGAGGACATCTTGTCTTCTTGACAATTAATCATCGGCTC | [symbol] |

Construction of base editor plasmids (MmuBE_E)

| | | |
|---|---|---|
| [symbol] | AAGCTTGGCTGTTTTGGCG | [symbol] |
| [symbol] | [symbol] | [symbol] |
| [symbol] | CAACTGCCCCCTCGAACCCCGGTGGAGGAGGTTCTGGAG | [symbol] |
| [symbol] | CGCCAAAACAGCCAAGCTTTTATGCAACCAGTCCTAGCATC | [symbol] |
| [symbol] | ACACGCTCTTCTATGACCGACGCTGAGTACGTG | [symbol] |
| [symbol] | ACACGCTCTTCTGGGGTTCGAGGGGGCAGTTG | [symbol] |
| [symbol] | [symbol] | [symbol] |
| | GCACCTGCACCAGCTCCAGCACCTGCTCCAGCTCCTGCTCCT | |
| [symbol] | [symbol] | [symbol] |
| | AGCAGGTGCTGGAGCAGGTGCTGGAGCAGGAGCTGGTGCAGG | |
| [symbol] | ACACGCTCTTCTCCCCTCCGGAGACTATAAGGACCAC | [symbol] |
| [symbol] | ACACGCTCTTCTTCATGGACTCGAGCCTAGACTTATC | [symbol] |
| [symbol] | ACACGCTCTTCTCCCCGGTGGAGGAGGTTCTGGAGG | [symbol] |
| [symbol] | ACACGCTCTTCCTTCATATACTTCTCCACGTAAGGGAC | [symbol] |
| [symbol] | ACACGCTCTTCTTCATTCCGGACTCGAGCCTAGACTT | [symbol] |
| [symbol] | ACACGAAGACTTCATCATGACAACAATGACAGTACATAC | [symbol] |
| [symbol] | ACACGAAGACTTTCATCCTAGACTTATCGTCATCG | [symbol] |
| [symbol] | ACACGAAGACTTATGAATGAGCTCAGAGACTGGCCC | [symbol] |
| [symbol] | ACACGAAGACTTCATCATGACAACAATGACAGTACATAC | [symbol] |
| [symbol] | ACACGAAGACAATGGGAACAGCAGGACTCTTAGTGG | [symbol] |

Construction of base editor plasmids (MmuBE_H)

| | | |
|---|---|---|
| [symbol] | CAAAGACGATGACGATAAGTCTAGGATGACAGACGCCGAGTACGTG | [symbol] |
| [symbol] | CCTCCACCTCCAGAACCTCCTCCACCCGGATTACTCGGTGCCGTGG | [symbol] |
| [symbol] | CCACGGCACCGAGTAATCCGGGTGGAGGAGGTTCTGGAGG | [symbol] |
| [symbol] | CACGTACTCGGCGTCTGTCAT | [symbol] |
| [symbol] | ACACGAAGACTTCATCATGACCACCATGACCGTGCAC | [symbol] |
| [symbol] | ACACGAAGACAACTGAGGTCCCGGGAGTCTCGCTGCCGCCGGATTACTCGGTGCCGTGG | [symbol] |
| [symbol] | [symbol] | [symbol] |
| [symbol] | ACACGAAGACAATGGGTTTCAACCCGGTGGCCCAG | [symbol] |
| [symbol] | [symbol] | [symbol] |
| [symbol] | ACACGAAGACAATGGGACCAACGGCTGGAGACTTAGTG | [symbol] |
| [symbol] | AGCGGCAGCGAGACTCCC | [symbol] |
| [symbol] | CGGATTACTCGGTGCCGTGG | [symbol] |

Construction of GFP silencing guide

| | | |
|---|---|---|
| [symbol] | AGACTTGAATTAGATGGTGATGTT | [symbol] |
| [symbol] | ACACAACATCACCATCTAATTCAA | [symbol] |

Construction of C-tile plasmids

| | | |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

Construction of C-motif plasmids

| | | |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

Separate sequencing primers

| | | |
|---|---|---|
| [symbol] | [symbol] | [symbol] |
| [symbol] | [symbol] | [symbol] |
| [symbol] | [symbol] | [symbol] |
| [symbol] | [symbol] | [symbol] |

S. cerevisiae

| | | |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| | | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

[symbol]

indicates data missing or illegible when filed

TABLE 2
plasmids and fragments used.
E. coli

| | | |
|---|---|---|
| pCMV-BE [symbol] | Cas9-APOBEC BE [symbol] under CMV promoter | addgene #73021 |
| pBI-Target-AID-NG | Cas9-CDA TargetAID | addgene #119861 |
| pSct_ [symbol] Cas9-CDA-UL | Proka [symbol] Cas9 Base editor | addgene #108551 |
| pCMV-dCpf1-BE | Cas12a-APOBEC base editor | addgene #107685 |
| pCMV-dCpf1-BE-YE | Cas12a-APOBEC(YE) base editor | addgene #107686 |
| pCas-dMmu | PJ23108-MmuciCas12u1 (E.coli harmonized) | chapter 6 |
| pCas-mRuby-UGI-Entry | mRuby flanked by Bbsl restriction sites for | this study |
| | cloning fusion proteins to UGI | |
| pCas-RFP-UGI-Entry | RFP flanked by Bbsl restriction sites for | this study |
| | cloning fusion proteins to UGI | |
| pCas-MmuBE_E1 | PJ23108-MmuciCas12u1-CDA-UGI | this study |
| | (121 as SH3 linker) | |
| pCas-MmuBE_E1.A | PJ23108-MmuciCas12u1-CDA-UGI | this study |
| | (96 as SH3 linker) | |
| pCas-MmuBE_E1.B | PJ23108-MmuciCas12u1-CDA-UGI | this study |
| | (67 as SH3 linker) | |
| pCas-MmuBE_E1.C | PJ23108-MmuciCas12u1-CDA-UGI | this study |
| | (24 as SH3 linker) | |
| pCas-MmuBE_E1.D | PJ23108-MmuciCas12u1-CDA-UGI | this study |
| | (33 as PAPA [symbol] linker) | |
| pCas-MmuBE_E2 | PJ23108-MmuciCas12u1-HsaAPOBEC-UGI | this study |
| | (93 as SH3 linker) | |
| pCas-MmuBE_E3 | PJ23108-MmuciCas12u1-CDA-HsaUGI | this study |
| | (121 as Sh3linker) | |
| pCas-MmuBE_ [symbol] | PJ23108-HsaMmuciCas12u1-HsaCDA-HsaUGI | this study |
| | (121 as SH3 linker) | |
| pCas-MmuBE_H1.A | PJ23108-HsaMmuciCas12u1-HsaAPOBEC-HsaUGI | this study |
| | ( [symbol] as XTEN linker) | |
| pCas-MmuBE_H2 | PJ23108-HsaMmuciCas12u1-HsaAPOBEC-HsaUGI | this study |
| | ( [symbol] as XTEN linker) | |
| pCas-MmuBE_H2YE | PJ23108-HsaMmuciCas12u1-HsaAPOBEC(YE)-HsaUGI | this study |
| | (121 as Sh3linker) | |
| pCRISPR-Mmu-NT (Bbsl) | PJ23119-CRISPR array (repeat-spacer-repeat). | this study |
| | 30 nt non-targetting spacer flanked by Bbsl | |
| pCRISPR-Mmu-NT | PJ23119-CRISPR array: non-targetting spacer (20 nt) | this study |
| pCRISPR-Mmu-GFP | PJ23119-CRISPR array: GFP spacer | this study |
| pCRISPR-Mmu-C-tile (WT) | PJ23119-CRISPR array: C-tile | this study |
| | (WT) spacer | |
| pCRISPR-Mmu-C-tile (1-6) | PJ23119-CRISPR array: C-tile | this study |
| | (1-6) spacer | |
| pCRISPR-Mmu-C-tile (4-9) | PJ23119-CRISPR array: C-tile | this study |
| | (4-9) spacer | |
| pCRISPR-Mmu-C-tile (7-12) | PJ23119-CRISPR array: C-tile | this study |
| | (7-12) spacer | |
| pCRISPR-Mmu-C-tile (10-15) | PJ23119-CRISPR array: C-tile | this study |
| | (10-15) spacer | |
| pCRISPR-Mmu-C-tile (13-18) | PJ23119-CRISPR array: C-tile | this study |
| | (13-18) spacer | |
| pCRISPR-Mmu-C-tile (16-20) | PJ23119-CRISPR array: C-tile | this study |
| | (16-20) spacer | |
| pCRISPR-Mmu-14 C-tile (WT) | PJ23119-CRISPR array: 14 nt | this study |
| | C-tile (WT) spacer | |
| pCRISPR-Mmu-15 C-tile (WT) | PJ23119-CRISPR array: 15 nt | this study |
| | C-tile (WT) spacer | |
| pCRISPR-Mmu-16 C-tile (WT) | PJ23119-CRISPR array: 16 nt | this study |
| | C-tile (WT) spacer | |
| pCRISPR-Mmu-17 C-tile (WT) | PJ23119-CRISPR array: 17 nt | this study |
| | C-tile (WT) spacer | |
| pCRISPR-Mmu-14 C-tile (10-15) | PJ23119-CRISPR array: 14 nt | this study |
| | C-tile (10-15) spacer | |
| pCRISPR-Mmu-15 C-tile (10-15) | PJ23119-CRISPR array: 15 nt | this study |
| | C-tile (10-15) spacer | |
| pCRISPR-Mmu-16 C-tile (10-15) | PJ23119-CRISPR array: 16 nt | this study |
| | C-tile (10-15) spacer | |

[symbol]

indicates data missing or illegible when filed

TABLE 3
S. cerevisiae strains used in the study.

| | | Obtained by | |
|---|---|---|---|
| Strain name | Genotype | transformation with | Origin |
| CEN.PK113-50 | MATa ura3-52 | | Euroscarf |
| YSTB013 | MATa ura3-52 pUDE731 | | This study |
| YSTB164 | MATa ura3-52 INT1::kl11p::eGFP::CYC1t | PL-098, A0135, | This study |
| | | A0136, A0195 | |
| | | and A0196 | |
| YSTB305 | MATa ura3-52 INT1::kl11p::eGFP::CYC1t, | A0246, A0247, | This study |
| | INT2::TEF1p::MmuCas12u1::CYC1t:: [symbol] | A0295, A0296 | |
| | | and A0297 | |
| YSTB315 | MATa ura3-52 INT1::kl11p::eGFP::CYC1t, | PL-242 | This study |
| | INT2::TEF1p::MmuCas12u1::CYC1t:: [symbol] + PL-242 | | |
| YSTB316 | MATa ura3-52 INT1::kl11p::eGFP::CYC1t, | PL-243 | This study |
| | INT2::TEF1p::MmuCas12u1::CYC1t:: [symbol] + PL-243 | | |
| YSTB317 | MATa ura3-52 INT1::kl11p::eGFP::CYC1t, | PL-244 | This study |
| | INT2::TEF1p::MmuCas12u1::CYC1t:: [symbol] + PL-244 | | |
| YSTB318 | MATa ura3-52 INT1::kl11p::eGFP::CYC1t, | PL-245 | This study |
| | INT2::TEF1p::MmuCas12u1::CYC1t:: [symbol] + PL-245 | | |
| YSTB319 | MATa ura3-52 INT1::kl11p::eGFP::CYC1t, | PL-246 | This study |
| | INT2::TEF1p::MmuCas12u1::CYC1t:: [symbol] + PL-246 | | |
| YSTB320 | MATa ura3-52 INT1::kl11p::eGFP::CYC1t, | PL-199 | This study |
| | INT2::TEF1p::MmuCas12u1::CYC1t:: [symbol] + PL-139 | | |

[symbol]

indicates data missing or illegible when filed

### Example 2

### Materials and methods

### Constructs

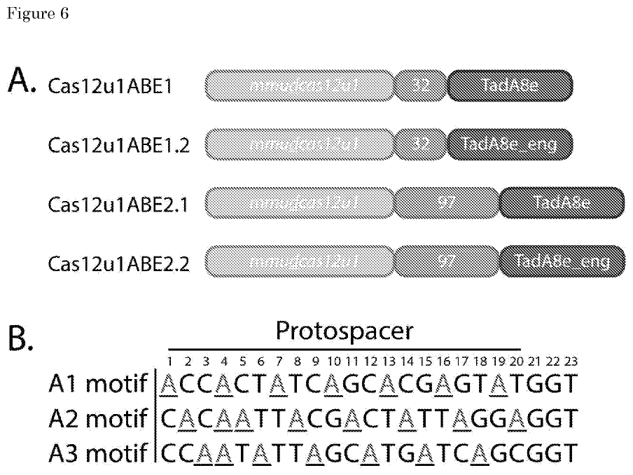

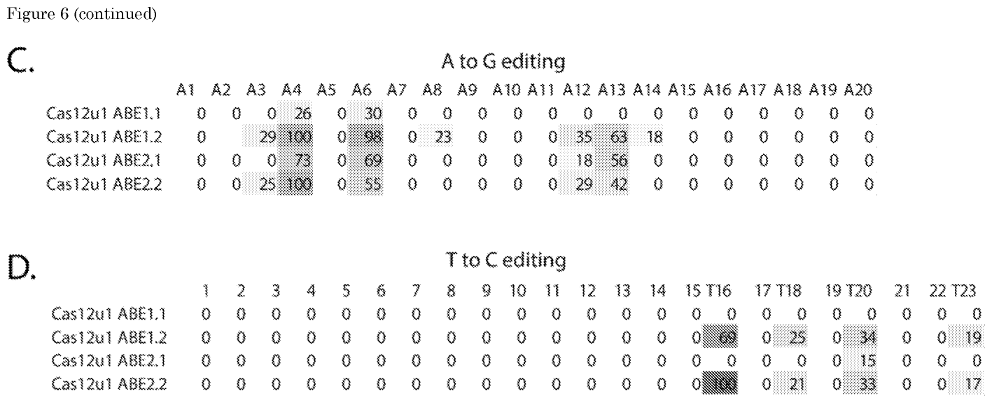

Adenine base editors (ABEs) were created by fusing small, catalytically inactive variants of Cas12 proteins to deaminase domains that were previously used for the Cas9 base editors. Specifically, at DNA level an engineered adenine deaminase TadA, called TadA8e, was covalently linked at the C-terminal end of either Cas12u1 or Cas12f1 (Richter et al., 2020. Nature Biotech 38: 883-891). These ABEs will be referred to as Cas12u1 ABE and Cas12f1 ABE. TadA8e is an engineered variant of the TadA protein (Richter et al., 2020. Nature Biotech 38: 883-891). Structural insights into TadA8e revealed that a loop at the C-terminus of TadA8e allowed for better accessibility to the non-target strand (NTS), thereby increasing editing efficiency in Cas9 and Cas12a ABEs (Lapinaite et al., 2020. Science 369: 566-571). However, in both Cas9 and Cas12a ABEs, the TadA8e was fused at the N-terminus of Cas9 and Cas12a. Therefore, to mimic this effect of better accessibility to the NTS in our Cas12u1 ABEs, we engineered TadA8e by placing the same loop found at the C-terminus now, with the amino acid residues in the reversed orientation, at the N-terminus. This engineered TadA8e is named TadA8e_eng (FIG. 5). In addition to modifying the adenine deaminase protein, two linkers with different composition and length were tested, an XXTEN (32 aa) linker and a shortened SH3 (93aa) linker (Gaudelli et al., 2017. Nature 551: 464-471; Banno et al., 2018. Nature Microbiol 3: 423-429). In total, four different Cas12u1 ABEs were constructed (FIG. 6A).

All ABEs were tested in E. coli using a three-plasmid system, consisting of pCas, pCRISPR and pTarget. pCas and pCRISPR expresses the ABE protein and guide RNA (20 nt spacer), respectively, whereas pTarget plasmids contain the protospacer/target sequence. We generated three variants of pTarget, also known as A motif plasmids, which contains a protospacer tiled A motif sequence (AxxAxxAxxAxxAxxAxxA), starting at every first (A1 motif), second (A2 motif) or third (A3 motif) nucleotide of the protospacer (FIG. 6B). Cells harboring all three plasmids for were grown for 72 hours and samples were taken at 24, 48 and 72 hours, and used for PCR amplification. Amplified PCR products were then analyzed by Sanger sequencing and base editing was determined by analyzing the obtained “mixed peaks” using the EditR program (Kluesner et al., 2018. CRISPR J 1: 239-250).

### Results

### Cas12U1

Results obtained from each A motif were combined for a better overview of the editing windows of the different Cas12u1 ABEs (FIGS. 6C and D). A to G base editing can be observed for all constructed Cas12u1 ABEs with relatively high A to G editing on position 4, 6, 12 and 13. Interestingly, increased A to G editing efficiency was found in constructs Cas12u1 ABE1.2 and Cas12u1 ABE2.2 which contained our engineered TadA8e_eng. This increased editing efficiency appeared to coincide with a broader editing window. Surprisingly, apart from an A to G base editing window on the PAM proximal end, an unprecedented T to C editing was detected at the PAM distal end. This implies that A to G editing occurs at the target strand instead of the expected non-target strand. T to C editing was mostly found in Cas12u1 ABE variants containing TadA8e_eng, Cas12u1 ABE1.2 and Cas12u1 ABE2.2. More specifically T to C editing was found on position, 16, 18, 20 and 23, of which 23 is outside the protospacer region. The tested protospacers did not contain a T on position 17, 19, 21 and 22, so T to C editing on these positions could not be tested. Future experiments will include protospacers containing T's in the rest of the PAM distal region, to resolve the T to C editing window for Cas12u1 ABEs.

### Cas12f1

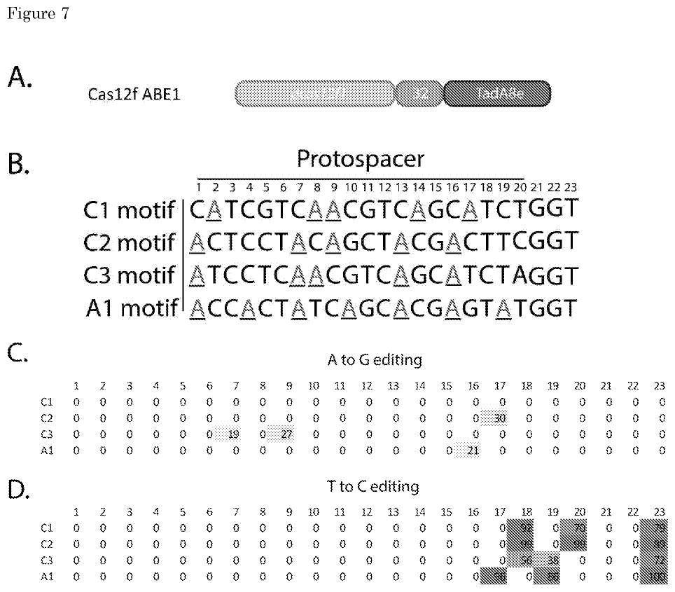

Next to Cas12u1 ABEs (˜2.4-2.6 kb), a smaller ABE was also constructed by fusing TadA8e to AsCas12f1, creating AsCas12f1 ABE1 (1.9 kb) (FIG. 7A). Protospacers used as targets for testing A to G base editing are C1, C2, C3 and A1 motif plasmids. These four plasmids cover adenine in total of positions on the 20 nt protospacer. Positions not included are positions 3, 5, 6, 11, 12, 15 and 18. Cells were grown for 72 hours harboring all three plasmids. Samples were used to PCR amplify the targeted region and analyzed by Sanger sequencing. Analysis of Sanger sequencing results by EditR, revealed successful A to G base editing on position, 7, 9, 16 and 17 by Cas12f1 ABEL Like Cas12u1 ABEs, Cas12f1 ABE1 also has an T to C editing window at the PAM distal end. This T to C editing window is currently estimated to be around 17-23, where position 23 is outside the targeted protospacer region.

In this work several small Cas12-based ABEs have been constructed showing successful A to G base editing in E. coli. Not only do these small Cas12 ABEs edit A to G (expected A to G base editing of the non-target strand), but they also unexpectedly edit T to C on the PAM distal end (A to G editing of the target strand). Base editing of the target strand has never been previously observed in other ABEs such as the one of Cas9 or Cas12a (Richter et al., 2020. Nature Biotech 38: 883-891). This can be due to the bulky size of the protein, that prevents access to the target. Another reason can be that the linkers used in Cas9 or Cas12a base editors do not allow proper position of the TadA8e to access the target strand. ABEs able to convert T to C increases the targeting scope of base editors and further expands the base editors toolbox. Currently both the Cas12u1 ABEs and the Cas12f1 ABE1 are the smallest adenine base editors known within the current ABE toolbox. These small ABEs hold great potential in application of human gene therapy as they easily fit into the AAV vector, allowing for AAV delivery in the human body.

### Example 3

### Materials and Methods

### Constructs

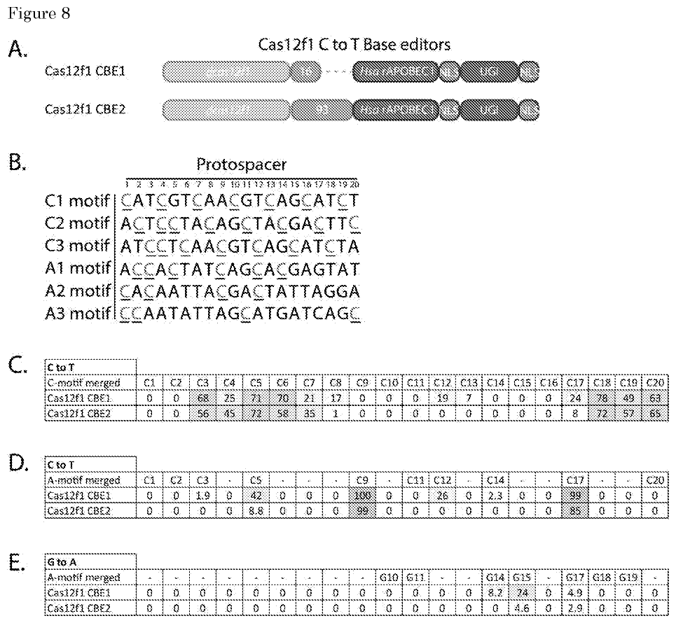

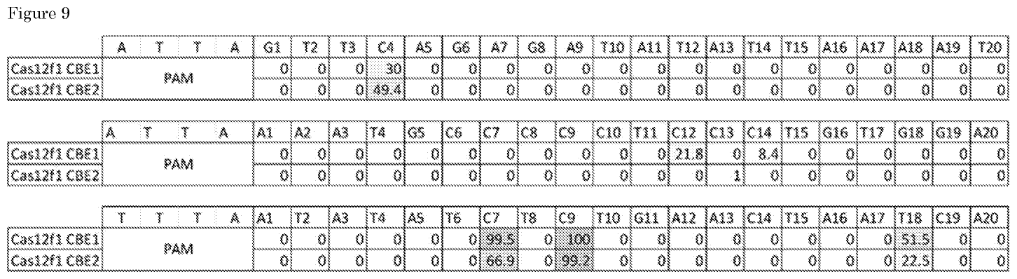

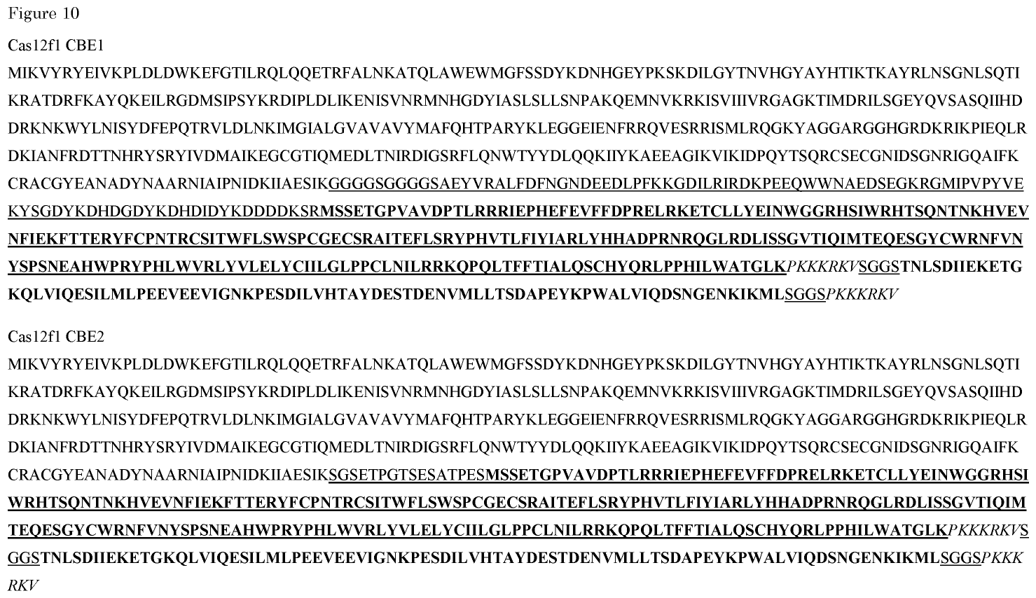

Cas12f1 (˜1.3 kb, 422 amino acids) from Acidibacillus sulfuroxidans (AsCas12f1), has been reported to recognize a 5′-YTTN PAM, wherein Y denotes a pyrimidine and N denotes any nucleotide), and to cleave dsDNA (Karvelis et al., 2020. Nucleic Acids Res 48: 5016-5023). Using gene synthesis, two AsCas12f1 cytosine base editors (CBEs) were constructed by fusing a catalytically inactive AsdCas12f1 (D225A) to a cytidine deaminase selected from CDA or rAPOBEC1 (apolipoprotein B mRNA editing enzyme, catalytic polypeptide-like) at the C-terminal end, using either a shortened SH3 (93 aa) or a XTEN (16 aa) linker, respectively (Komor et al., 2016. Nature 533: 420-424; Banno et al., 2018. Nature Microbiol 3: 423-429). (See FIG. 8). In addition to cytidine deaminases, an UGI domain and a nuclear localization signal (NLS) were added. These Cas12f1 CBEs are named Cas12f1 CBE1 and Cas12f1 CBE2 (FIG. 8A and FIG. 9).

### Results

All ABEs were tested in E. coli using a three-plasmid system, consisting of pCas, pCRISPR and pTarget. pCas and pCRISPR expresses the ABE protein and guide RNA (20 nt spacer), respectively, whereas pTarget plasmids contain the protospacer target sequence. We generated two sets, with each set containing three variants of a pTarget plasmid, also known as C motif and A motif plasmids, which contains a protospacer tiled with C or A motif sequence (CxxCxxCxxCxxCxxCxxC or AxxAxxAxxAxxAxxAxxA), starting at every first (C1/A1 motif), second (C2/A2 motif) or third (C3/A3 motif) nucleotide of the protospacer (FIG. 8B). C motif protospacers contain a 5′-TTTA PAM and A motif protospacers contain a 5′-CTTA PAM. A motif plasmids were initially designed to test A to G base editors, but were also used for testing CBEs in order to obtain more data regarding base editing efficiencies and windows. Cells harboring all three plasmids were grown for 72 hours and were used for PCR amplification. Amplified PCR products were then sequenced by Sanger sequencing and base editing was determined by analyzing the obtained “mixed peaks” using EditR. Results obtained from both C motif and A motif plasmids were combined for a better overview of the editing windows of the different Cas12f1 CBEs (FIGS. 8C and D). Both Cas12f1 CBEs have a wide C to T editing window that consists of two regions, a PAM proximal (˜3-9 nucleotides) and a PAM distal (-17-20 nucleotides) region. In addition to the two regions, Cas12f1 CBE1 also edits the middle region (12-14 nucleotides; see FIG. 8C, D, E).

Surprisingly, analysis of the non-target strand revealed, aside from C to T editing, an unprecedented G to A editing (actually: C to T editing of the target strand) was also observed in both Cas12f1 CBEs, with the most edited position being G15 (see FIG. 8D). Cas12f1 CBE1 exhibited higher G to A editing and contains a larger editing window (editing especially nucleotide positions 14-15 and 17), when compared to Cas12f1 CBE2 (editing especially nucleotide positions 15 and 17). Though Cas12f1 CBE1 contains a shorter linker, it was found to exhibit a higher editing efficiency as well as a wider editing window. This suggests the importance of linkers in fine tuning base editing windows.

### Example 4

### Materials and Methods

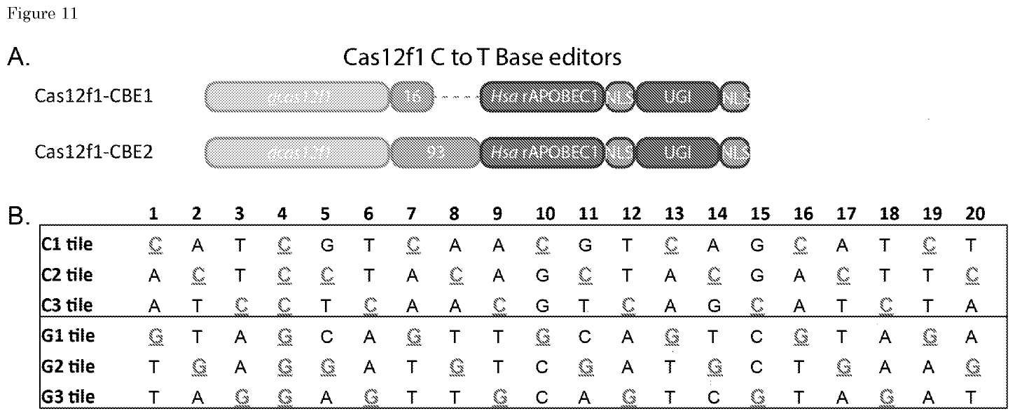

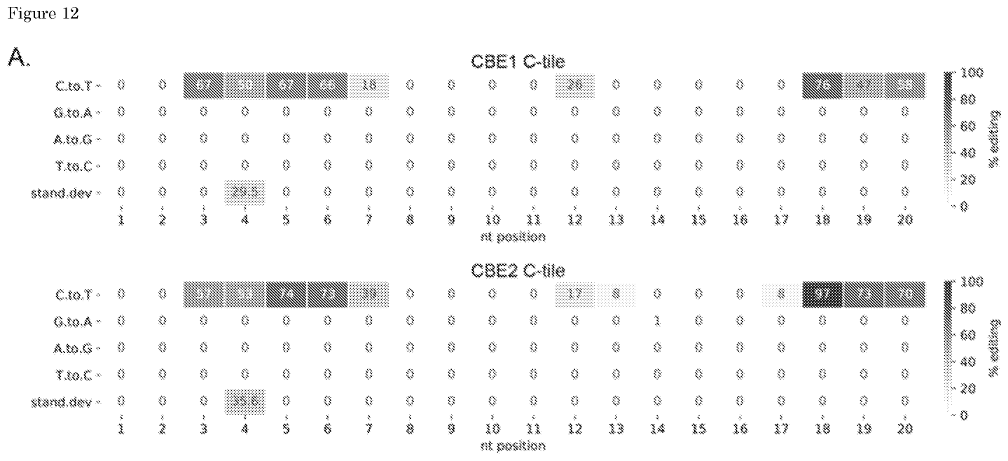

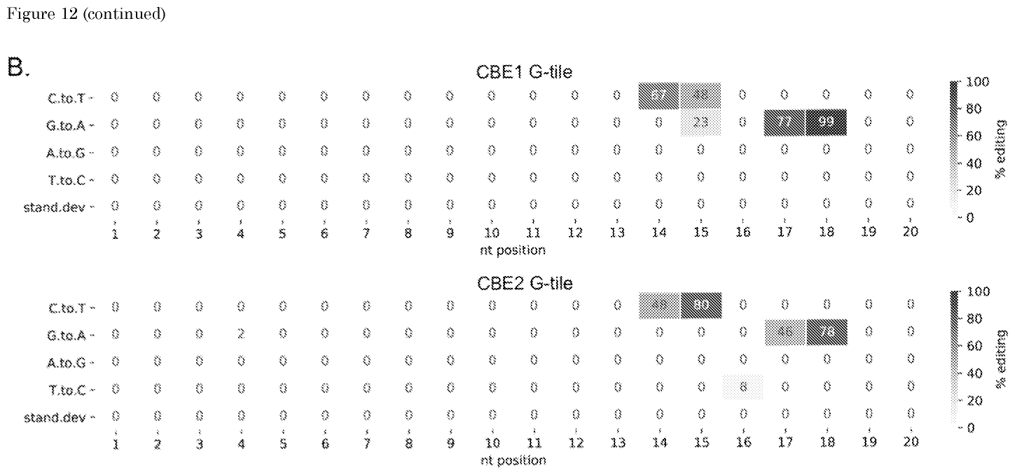

As is shown in Example 3 herein above, we have developed two cytidine base editors (CBE) using a catalytically inactive Cas12f1 (dCas12f1). dCas12f1 was fused to a cytidine deaminase (rAPOBEC1) using two different linkers resulting in Cas12f1-CBE1 and Cas12f1-CBE2. Cas12f1-CBE1 has an XTEN linker (16 aa) and Cas12f1-CBE2 an SH3 linker (Komor et al., 2016. Nature 533: 420-424; Nishida et al., 2016. Science 353: 1248-aaf8729) (See FIG. 11A). To determine the editing location within the targeted region (base editing window), we used a set of pTarget plasmids that contain the protospacer target sequenced. We generated two sets of pTarget plasmids, C-tile and G-tile, of which each set consists of three variants. The C/G-tile plasmids contain a tiled C/G motif (CxxCxxCxxCxxCxxCxxC or GxxGxxGxxGxxGxxGxxG), starting at every first (C/G1), second (C/G2) or third (C/G3) nucleotide of the protospacer (FIG. 11B). E. coli cells containing pCas (expressing Cas12f1-CBE1/2), pCRISPR(expressing CRISPR-array) and pTarget tile plasmids were grown for 48 hours and were used for a population PCR, which amplified the protospacer region on the tile plasmids. Amplified products were sent for Sanger Sequencing and results were analyzed by EditR (Kluesner et al., 2018. CRISPR J 1: 239-250). Base editing results obtained from all three tile plasmids were merged and visualized in a heatmap (FIG. 12).

### Results

Both Cas12f1-CBEs have a C to T base editing window on the non-target strand, as has previously been demonstrated for Cas9-CBE and Cas12a-CBE (see Example 3). Remarkably, the base editing window of the Cas12f1-CBEs consists of two regions, a PAM-proximal region (3-7) and a PAM-distal region (18-20) (FIG. 12A). Small amounts of editing on position 12 can also be observed for both CBEs. Surprisingly, in addition to editing the non-target strand, Cas12f1-CBEs is also able to edit the target strand, resulting in G to A conversion on the non-target strand. Both Cas12f1-CBEs edit positions 17-18 on the target strand, while Cas12f1-CBE1 also edits position 15 on the target strand, resulting in a G to A conversion on this position on the non-target strand (FIG. 12B). In case of the CBE2 G-tile (FIG. 12B, lower panel), occasionally sequences are observed that suggest T to C editing (e.g. positions 4 and 16 for Cas12f1-CBE2); this can only be explained by sequencing noise, and should be ignored.

1. A method for base-editing a double stranded target nucleic acid (dstna) in a cell, comprising providing the cell with a clustered regularly interspaced short palindromic repeat (CRISPR)-based editing system comprising a guide ribonucleic acid (gRNA) of which a part is complementary to a nucleic acid strand of a dstna, and a cleavage-deficient CRISPR-associated (Cas) nuclease that is fused to a deaminase, whereby the editing system provides A to G editing, and/or C to T editing, of the complementary strand of the dstna.

2. The method of claim 1, wherein the deaminase comprises an adenine and/or a cytidine deamination enzyme.

3. The method of claim 2, wherein the cytidine deamination enzyme, the adenine deamination enzyme, or both, are fused to the C-terminus of the Cas nuclease.

4. The method of claim 1, wherein the Cas nuclease lacks at least part of a recognition lobe, termed Rec1 and Rec2 domain, when compared to other Cas nucleases.

5. The method of claim 1, wherein Cas nuclease comprises at most 800 amino acid residues.

6. The method of claim 1, wherein the Cas nuclease multimerizes upon binding to the gRNA in the CRISPR-based editing system, preferably dimerizes.

7. The method of claim 1, wherein the Cas nuclease is a C2c4 nuclease, or a Cas12F nuclease.

8. The method of claim 1, wherein the Cas nuclease is fused to a deaminase, whereby a loop structure at the C-terminal region of the deaminase is duplicated at the N-terminal region of the deaminase, and a C-terminal helix structure is duplicated and inverted as N-terminus of the deaminase.

9. The method of claim 1, wherein the Cas nuclease is fused to a deaminase through a linker sequence.

10. A nucleotide molecule encoding a Cas nuclease that is fused to a deaminase, allowing A to G editing and/or C to T editing of the complementary strand of the ds target nucleic acid, as depicted in claim 1.

11. An expression vector comprising the nucleotide molecule of claim 10, under control of a suitable expression promoter.

12. The expression vector of claim 11, further comprising a nucleotide sequence comprising an expression promoter and a sequence under the control of the promoter encoding a gRNA comprising a nucleotide sequence which recognises a target locus of interest.

13. A cell comprising the expression vector of claim 11.

14. An isolated Cas nuclease that is fused to an adenine deaminase and/or cytidine deaminase, allowing A to G editing and/or C to T editing, of the complementary strand of the ds target nucleic acid, as depicted in claim 1.

15. The isolated Cas nuclease according to claim 14, wherein a adenine or cytidine deamination enzyme is fused to the C-terminus of the Cas nuclease, optionally wherein a cytidine deamination enzyme is fused to the N-terminal end of the Cas nuclease and the adenine deamination enzyme is fused to the C-terminal end.


SEQUENCE LISTING (229 sequences)

SEQ ID NO: 1 - protein, 10 aa, Artificial Sequence
  c-myc domain tag
EQKLISEEDL

SEQ ID NO: 2 - protein, 9 aa, Artificial Sequence
  hemagglutinin tag
YPYDVPDYA

SEQ ID NO: 3 - protein, 12 aa, Artificial Sequence
  C-terminal loop domain
FNAQKKAQSSIN

SEQ ID NO: 4 - protein, 12 aa, Artificial Sequence
  C-terminal loop domain in inverted orientation
NISSQAKKQANF

SEQ ID NO: 5 - protein, 6 aa, Artificial Sequence
  loop region
YRMPRQ

SEQ ID NO: 6 - protein, 18 aa, Artificial Sequence
  amino acid sequence
NISSQAKKQANFYRMPRQ

SEQ ID NO: 7 - DNA, 19 bp, Artificial Sequence
  C motif sequence
  misc_feature (2)..(3): n is a, c, g, or t
  misc_feature (5)..(6): n is a, c, g, or t
  misc_feature (8)..(9): n is a, c, g, or t
  misc_feature (11)..(12): n is a, c, g, or t
  misc_feature (14)..(15): n is a, c, g, or t
  misc_feature (17)..(18): n is a, c, g, or t
cnncnncnncnncnncnnc

SEQ ID NO: 8 - DNA, 19 bp, Artificial Sequence
  oligonucleotide BG14064
gatgtcctcctgagctcgc

SEQ ID NO: 9 - DNA, 19 bp, Artificial Sequence
  oligonucleotide BG14065
aagcttggctgttttggcg

SEQ ID NO: 10 - DNA, 40 bp, Artificial Sequence
  oligonucleotide BG19000
acgagctgtacaagactagtcccaagaagaaacggaaagt

SEQ ID NO: 11 - DNA, 41 bp, Artificial Sequence
  oligonucleotide BG19001
cgccaaaacagccaagcttttagactttcctcttcttcttg

SEQ ID NO: 12 - DNA, 40 bp, Artificial Sequence
  oligonucleotide BG19002
gcgagctcaggaggaccatatggtgtctaagggcgaagag

SEQ ID NO: 13 - DNA, 26 bp, Artificial Sequence
  oligonucleotide BG19003
actagtcttgtacagctcgtccatgc

SEQ ID NO: 14 - DNA, 20 bp, Artificial Sequence
  oligonucleotide BG19102
cccaagaagaaacggaaagt

SEQ ID NO: 15 - DNA, 48 bp, Artificial Sequence
  oligonucleotide BG19104
cactttccgtttcttcttgggaagtcttcgttaagcaccggtggagtg

SEQ ID NO: 16 - DNA, 48 bp, Artificial Sequence
  oligonucleotide BG19118
gcgagctcaggaggacatcttgtcttcttgacaattaatcatcggctc

SEQ ID NO: 17 - DNA, 19 bp, Artificial Sequence
  oligonucleotide BG14065
aagcttggctgttttggcg

SEQ ID NO: 18 - DNA, 20 bp, Artificial Sequence
  oligonucleotide BG15295
ggggttcgagggggcagttg

SEQ ID NO: 19 - DNA, 39 bp, Artificial Sequence
  oligonucleotide BG15296
caactgccccctcgaaccccggtggaggaggttctggag

SEQ ID NO: 20 - DNA, 41 bp, Artificial Sequence
  oligonucleotide BG15297
cgccaaaacagccaagcttttatgcaaccagtcctagcatc

SEQ ID NO: 21 - DNA, 33 bp, Artificial Sequence
  oligonucleotide BG20896
acacgctcttctatgaccgacgctgagtacgtg

SEQ ID NO: 22 - DNA, 32 bp, Artificial Sequence
  oligonucleotide BG20897
acacgctcttctggggttcgagggggcagttg

SEQ ID NO: 23 - DNA, 115 bp, Artificial Sequence
  oligonucleotide BG20898
acacgctcttctcccccctgcaccagctcctgctccagcacctgctccagcacctgctccagctccagcacctgcacctgcaccagctccagcacctgctccagctcctgctcct

SEQ ID NO: 24 - DNA, 115 bp, Artificial Sequence
  oligonucleotide BG20899
acacgctcttcttcataggagcaggagctggagcaggtgctggagctggtgcaggtgcaggtgctggagctggagcaggtgctggagcaggtgctggagcaggagctggtgcagg

SEQ ID NO: 25 - DNA, 37 bp, Artificial Sequence
  oligonucleotide BG20900
acacgctcttctcccctccggagactataaggaccac

SEQ ID NO: 26 - DNA, 37 bp, Artificial Sequence
  oligonucleotide BG20901
acacgctcttcttcatggactcgagcctagacttatc

SEQ ID NO: 27 - DNA, 36 bp, Artificial Sequence
  oligonucleotide BG20902
acacgctcttctccccggtggaggaggttctggagg

SEQ ID NO: 28 - DNA, 37 bp, Artificial Sequence
  oligonucleotide BG20903
acacgctcttcttcatatacttctccacgtaagggac

SEQ ID NO: 29 - DNA, 37 bp, Artificial Sequence
  oligonucleotide BG20904
acacgctcttcttcattccggactcgagcctagactt

SEQ ID NO: 30 - DNA, 39 bp, Artificial Sequence
  oligonucleotide BG20905
acacgaagacttcatcatgacaacaatgacagtacatac

SEQ ID NO: 31 - DNA, 35 bp, Artificial Sequence
  oligonucleotide BG20906
acacgaagactttcatcctagacttatcgtcatcg

SEQ ID NO: 32 - DNA, 36 bp, Artificial Sequence
  oligonucleotide BG20907
acacgaagacttatgaatgagctcagagactggccc

SEQ ID NO: 33 - DNA, 39 bp, Artificial Sequence; identical to SEQ ID NO: 30
  oligonucleotide BG20908

SEQ ID NO: 34 - DNA, 36 bp, Artificial Sequence
  oligonucleotide BG20909
acacgaagacaatgggaacagcaggactcttagtgg

SEQ ID NO: 35 - DNA, 46 bp, Artificial Sequence
  oligonucleotide BG21058
caaagacgatgacgataagtctaggatgacagacgccgagtacgtg

SEQ ID NO: 36 - DNA, 45 bp, Artificial Sequence
  oligonucleotide BG21059
ctccacctccagaacctcctccacccggattactcggtgccgtgg

SEQ ID NO: 37 - DNA, 40 bp, Artificial Sequence
  oligonucleotide BG21060
ccacggcaccgagtaatccgggtggaggaggttctggagg

SEQ ID NO: 38 - DNA, 21 bp, Artificial Sequence
  oligonucleotide BG21061
cacgtactcggcgtctgtcat

SEQ ID NO: 39 - DNA, 37 bp, Artificial Sequence
  oligonucleotide BG19700
acacgaagacttcatcatgaccaccatgaccgtgcac

SEQ ID NO: 40 - DNA, 59 bp, Artificial Sequence
  oligonucleotide BG19702
acacgaagacaactgaggtcccgggagtctcgctgccgccggattactcggtgccgtgg

SEQ ID NO: 41 - DNA, 58 bp, Artificial Sequence
  oligonucleotide BG19703
acacgaagactttcagagtccgccacacccgaaagtatgagctcagagactggcccag

SEQ ID NO: 42 - DNA, 35 bp, Artificial Sequence
  oligonucleotide BG19704
acacgaagacaatgggtttcaacccggtggcccag

SEQ ID NO: 43 - DNA, 57 bp, Artificial Sequence
  oligonucleotide BG19709
acacgaagactttcagagtccgccacacccgaaagtatgacagacgccgagtacgtg

SEQ ID NO: 44 - DNA, 38 bp, Artificial Sequence
  oligonucleotide BG19710
acacgaagacaatgggaccaacggctggagacttagtg

SEQ ID NO: 45 - DNA, 18 bp, Artificial Sequence
  oligonucleotide BG21160
agcggcagcgagactccc

SEQ ID NO: 46 - DNA, 20 bp, Artificial Sequence
  oligonucleotide BG21161
cggattactcggtgccgtgg

SEQ ID NO: 47 - DNA, 24 bp, Artificial Sequence
  oligonucleotide BG20003
agacttgaattagatggtgatgtt

SEQ ID NO: 48 - DNA, 24 bp, Artificial Sequence
  oligonucleotide BG20004
acacaacatcaccatctaattcaa

SEQ ID NO: 49 - DNA, 34 bp, Artificial Sequence
  oligonucleotide BG15892
gatcggtacccattaacctataaaaataggcgtc

SEQ ID NO: 50 - DNA, 23 bp, Artificial Sequence
  oligonucleotide BG15893
caatttttaatgaacggacgtcg

SEQ ID NO: 51 - DNA, 29 bp, Artificial Sequence
  oligonucleotide BG15894
cttattcattaaaaattgaattgaggtac

SEQ ID NO: 52 - DNA, 29 bp, Artificial Sequence
  oligonucleotide BG15895
ctcaattcaatttttaatgaataagacgt

SEQ ID NO: 53 - DNA, 29 bp, Artificial Sequence
  oligonucleotide BG15896
cttaccccccaaaaattgaattgaggtac

SEQ ID NO: 54 - DNA, 29 bp, Artificial Sequence
  oligonucleotide BG15897
ctcaattcaatttttggggggtaagacgt

SEQ ID NO: 55 - DNA, 29 bp, Artificial Sequence
  oligonucleotide BG15898
cttattcccccccaattgaattgaggtac

SEQ ID NO: 56 - DNA, 29 bp, Artificial Sequence
  oligonucleotide BG15899
ctcaattcaattgggggggaataagacgt

SEQ ID NO: 57 - DNA, 29 bp, Artificial Sequence
  oligonucleotide BG15900
cttattcattcccccctgaattgaggtac

SEQ ID NO: 58 - DNA, 29 bp, Artificial Sequence
  oligonucleotide BG15901
ctcaattcaggggggaatgaataagacgt

SEQ ID NO: 59 - DNA, 29 bp, Artificial Sequence
  oligonucleotide BG15902
cttattcattaaaccccccattgaggtac

SEQ ID NO: 60 - DNA, 29 bp, Artificial Sequence
  oligonucleotide BG15903
ctcaatggggggtttaatgaataagacgt

SEQ ID NO: 61 - DNA, 29 bp, Artificial Sequence
  oligonucleotide BG15904
cttattcattaaaaatccccccgaggtac

SEQ ID NO: 62 - DNA, 29 bp, Artificial Sequence
  oligonucleotide BG15905
ctcggggggatttttaatgaataagacgt

SEQ ID NO: 63 - DNA, 29 bp, Artificial Sequence
  oligonucleotide BG15906
cttattcattaaaaattgacccccggtac

SEQ ID NO: 64 - DNA, 29 bp, Artificial Sequence
  oligonucleotide BG15907
cgggggtcaatttttaatgaataagacgt

SEQ ID NO: 65 - DNA, 24 bp, Artificial Sequence
  oligonucleotide BG15908
agacttcattaaaaattgaattga

SEQ ID NO: 66 - DNA, 24 bp, Artificial Sequence
  oligonucleotide BG15909
acactcaattcaatttttaatgaa

SEQ ID NO: 67 - DNA, 24 bp, Artificial Sequence
  oligonucleotide BG15910
agacccccccaaaaattgaattga

SEQ ID NO: 68 - DNA, 24 bp, Artificial Sequence
  oligonucleotide BG15911
acactcaattcaatttttgggggg

SEQ ID NO: 69 - DNA, 24 bp, Artificial Sequence
  oligonucleotide BG15912
agacttcccccccaattgaattga

SEQ ID NO: 70 - DNA, 24 bp, Artificial Sequence
  oligonucleotide BG15913
acactcaattcaattgggggggaa

SEQ ID NO: 71 - DNA, 24 bp, Artificial Sequence
  oligonucleotide BG15914
agacttcattcccccctgaattga

SEQ ID NO: 72 - DNA, 24 bp, Artificial Sequence
  oligonucleotide BG15915
acactcaattcaggggggaatgaa

SEQ ID NO: 73 - DNA, 24 bp, Artificial Sequence
  oligonucleotide BG15916
agacttcattaaaccccccattga

SEQ ID NO: 74 - DNA, 24 bp, Artificial Sequence
  oligonucleotide BG15917
acactcaatggggggtttaatgaa

SEQ ID NO: 75 - DNA, 24 bp, Artificial Sequence
  oligonucleotide BG15918
agacttcattaaaaatccccccga

SEQ ID NO: 76 - DNA, 24 bp, Artificial Sequence
  oligonucleotide BG15919
acactcggggggatttttaatgaa

SEQ ID NO: 77 - DNA, 24 bp, Artificial Sequence
  oligonucleotide BG15920
agacttcattaaaaattgaccccc

SEQ ID NO: 78 - DNA, 24 bp, Artificial Sequence
  oligonucleotide BG15921
acacgggggtcaatttttaatgaa

SEQ ID NO: 79 - DNA, 18 bp, Artificial Sequence
  oligonucleotide BG18520
agacttcattaaaaattg

SEQ ID NO: 80 - DNA, 18 bp, Artificial Sequence
  oligonucleotide BG18521
acaccaatttttaatgaa

SEQ ID NO: 81 - DNA, 19 bp, Artificial Sequence
  oligonucleotide BG18522
agacttcattaaaaattga

SEQ ID NO: 82 - DNA, 19 bp, Artificial Sequence
  oligonucleotide BG18523
acactcaatttttaatgaa

SEQ ID NO: 83 - DNA, 20 bp, Artificial Sequence
  oligonucleotide BG18524
agacttcattaaaaattgaa

SEQ ID NO: 84 - DNA, 20 bp, Artificial Sequence
  oligonucleotide BG18525
acacttcaatttttaatgaa

SEQ ID NO: 85 - DNA, 21 bp, Artificial Sequence
  oligonucleotide BG18526
agacttcattaaaaattgaat

SEQ ID NO: 86 - DNA, 21 bp, Artificial Sequence
  oligonucleotide BG18527
acacattcaatttttaatgaa

SEQ ID NO: 87 - DNA, 18 bp, Artificial Sequence
  oligonucleotide BG18528
agacttcattaaaccccc

SEQ ID NO: 88 - DNA, 18 bp, Artificial Sequence
  oligonucleotide BG18529
acacgggggtttaatgaa

SEQ ID NO: 89 - DNA, 19 bp, Artificial Sequence
  oligonucleotide BG18530
agacttcattaaacccccc

SEQ ID NO: 90 - DNA, 19 bp, Artificial Sequence
  oligonucleotide BG18531
acacggggggtttaatgaa

SEQ ID NO: 91 - DNA, 20 bp, Artificial Sequence
  oligonucleotide BG18532
agacttcattaaacccccca

SEQ ID NO: 92 - DNA, 20 bp, Artificial Sequence
  oligonucleotide BG18533
acactggggggtttaatgaa

SEQ ID NO: 93 - DNA, 21 bp, Artificial Sequence
  oligonucleotide BG18534
agacttcattaaaccccccat

SEQ ID NO: 94 - DNA, 21 bp, Artificial Sequence
  oligonucleotide BG18535
acacatggggggtttaatgaa

SEQ ID NO: 95 - DNA, 18 bp, Artificial Sequence
  oligonucleotide BG18536
agacttcattaaaaatcc

SEQ ID NO: 96 - DNA, 18 bp, Artificial Sequence
  oligonucleotide BG18537
acacggatttttaatgaa

SEQ ID NO: 97 - DNA, 19 bp, Artificial Sequence
  oligonucleotide BG18538
agacttcattaaaaatccc

SEQ ID NO: 98 - DNA, 19 bp, Artificial Sequence
  oligonucleotide BG18539
acacgggatttttaatgaa

SEQ ID NO: 99 - DNA, 20 bp, Artificial Sequence
  oligonucleotide BG18540
agacttcattaaaaatcccc

SEQ ID NO: 100 - DNA, 20 bp, Artificial Sequence
  oligonucleotide BG18541
acacggggatttttaatgaa

SEQ ID NO: 101 - DNA, 21 bp, Artificial Sequence
  oligonucleotide BG18542
agacttcattaaaaatccccc

SEQ ID NO: 102 - DNA, 21 bp, Artificial Sequence
  oligonucleotide BG18543
acacgggggatttttaatgaa

SEQ ID NO: 103 - DNA, 20 bp, Artificial Sequence
  oligonucleotide BG18544
agacttcattaaaaattgac

SEQ ID NO: 104 - DNA, 20 bp, Artificial Sequence
  oligonucleotide BG18545
acacgtcaatttttaatgaa

SEQ ID NO: 105 - DNA, 21 bp, Artificial Sequence
  oligonucleotide BG18546
agacttcattaaaaattgacc

SEQ ID NO: 106 - DNA, 21 bp, Artificial Sequence
  oligonucleotide BG18547
acacggtcaatttttaatgaa

SEQ ID NO: 107 - DNA, 24 bp, Artificial Sequence
  oligonucleotide BG20162
agaccatcgtcaacgtcagcatct

SEQ ID NO: 108 - DNA, 24 bp, Artificial Sequence
  oligonucleotide BG20163
acacagatgctgacgttgacgatg

SEQ ID NO: 109 - DNA, 24 bp, Artificial Sequence
  oligonucleotide BG20164
agacactgctacagctacgacttc

SEQ ID NO: 110 - DNA, 24 bp, Artificial Sequence
  oligonucleotide BG20165
acacgaagtcgtagctgtagcagt

SEQ ID NO: 111 - DNA, 24 bp, Artificial Sequence
  oligonucleotide BG20166
agacatcgtcaacgtcagcatcta

SEQ ID NO: 112 - DNA, 24 bp, Artificial Sequence
  oligonucleotide BG20167
acactagatgctgacgttgacgat

SEQ ID NO: 113 - DNA, 29 bp, Artificial Sequence
  oligonucleotide BG20168
cttacatcgtcaacgtcagcatctggtac

SEQ ID NO: 114 - DNA, 29 bp, Artificial Sequence
  oligonucleotide BG20169
cagatgctgacgttgacgatgtaagacgt

SEQ ID NO: 115 - DNA, 29 bp, Artificial Sequence
  oligonucleotide BG20170
cttaactgctacagctacgacttcggtac

SEQ ID NO: 116 - DNA, 29 bp, Artificial Sequence
  oligonucleotide BG20171
cgaagtcgtagctgtagcagttaagacgt

SEQ ID NO: 117 - DNA, 29 bp, Artificial Sequence
  oligonucleotide BG20172
cttaatcgtcaacgtcagcatctaggtac

SEQ ID NO: 118 - DNA, 29 bp, Artificial Sequence
  oligonucleotide BG20173
ctagatgctgacgttgacgattaagacgt

SEQ ID NO: 119 - DNA, 20 bp, Artificial Sequence
  oligonucleotide BG15302
ggtgatgtcggcgatatagg

SEQ ID NO: 120 - DNA, 27 bp, Artificial Sequence
  oligonucleotide BG15034
gatttaatctgtatcaggctgaaaatc

SEQ ID NO: 121 - DNA, 23 bp, Artificial Sequence
  oligonucleotide BG18776
cttgatcagatcttgatcccctg

SEQ ID NO: 122 - DNA, 24 bp, Artificial Sequence
  oligonucleotide BG18664
acaccttcaccctctccactgaca

SEQ ID NO: 123 - DNA, 20 bp, Artificial Sequence
  oligonucleotide BG11655
cgttggatctctcttctaag

SEQ ID NO: 124 - DNA, 20 bp, Artificial Sequence
  oligonucleotide BG11656
cacctgtaagcgttgatttc

SEQ ID NO: 125 - DNA, 24 bp, Artificial Sequence
  oligonucleotide BG12945
gatcatttatctttcactgcggag

SEQ ID NO: 126 - DNA, 29 bp, Artificial Sequence
  oligonucleotide BG16493
tttttttgttttttatgtcttatcgtgac

SEQ ID NO: 127 - DNA, 46 bp, Artificial Sequence
  oligonucleotide BG14031
tactgttgtagattttttttgttttttatgtcttatcgtgacgcag

SEQ ID NO: 128 - DNA, 48 bp, Artificial Sequence
  oligonucleotide BG14032
agacataaaaaacaaaaaaaatctacaacagtagaaatttccacacca

SEQ ID NO: 129 - DNA, 49 bp, Artificial Sequence
  oligonucleotide BG16236
tctcccaccagcaaagcctgaatttctactgttgtagattttttttgtt

SEQ ID NO: 130 - DNA, 25 bp, Artificial Sequence
  oligonucleotide BG14555
atctacaacagtagaaattgatcat

SEQ ID NO: 131 - DNA, 30 bp, Artificial Sequence
  oligonucleotide BG16700
cgccggcgcgatgccgccaaaacgtcttgg

SEQ ID NO: 132 - DNA, 20 bp, Artificial Sequence
  oligonucleotide BG14156
ggtgatgtcggcgatatagg

SEQ ID NO: 133 - DNA, 23 bp, Artificial Sequence
  oligonucleotide BG17373
cactatagcaatctggctatatg

SEQ ID NO: 134 - DNA, 70 bp, Artificial Sequence
  oligonucleotide BG17374
aaacgcctgtgggtgtggtactggatatgcaaagcgattggaagtcgcttgactcctctgccgtcattcc

SEQ ID NO: 135 - DNA, 72 bp, Artificial Sequence
  oligonucleotide BG17375
ttgcccatcgaacgtacaagtactcctctgttctctccttcctttgctttaagcgttgaagtttcctctttg

SEQ ID NO: 136 - DNA, 20 bp, Artificial Sequence
  oligonucleotide BG17376
tgtcaactggagagctatcg

SEQ ID NO: 137 - DNA, 24 bp, Artificial Sequence
  oligonucleotide BG18470
agacgcgttgagttggtgtatggc

SEQ ID NO: 138 - DNA, 24 bp, Artificial Sequence
  oligonucleotide BG18471
acacgccatacaccaactcaacgc

SEQ ID NO: 139 - DNA, 74 bp, Artificial Sequence
  oligonucleotide BG19774
aagcgacttccaatcgctttgcatatccagtaccacacccacaggcgtttttttctttttttgcggtcaccccc

SEQ ID NO: 140 - DNA, 55 bp, Artificial Sequence
  oligonucleotide BG19775
tgaaaagttcttctcctttgctcatttttgataagtatttaagcgagtgactgaa

SEQ ID NO: 141 - DNA, 54 bp, Artificial Sequence
  oligonucleotide BG19776
tcactcgcttaaatacttatcaaaaatgagcaaaggagaagaacttttcactgg

SEQ ID NO: 142 - DNA, 71 bp, Artificial Sequence
  oligonucleotide BG17378
aagcaaaggaaggagagaacagaggagtacttgtacgttcgatgggcaacttcgagcgtcccaaaaccttc

SEQ ID NO: 143 - DNA, 18 bp, Artificial Sequence
  oligonucleotide BG20455
accgctgctccccgtagc

SEQ ID NO: 144 - DNA, 25 bp, Artificial Sequence
  oligonucleotide BG13880
tgcttcattttgtagaacaaaaatg

SEQ ID NO: 145 - DNA, 31 bp, Artificial Sequence
  oligonucleotide BG19061
agacaccgtgcataccgctgctccccgtagc

SEQ ID NO: 146 - DNA, 31 bp, Artificial Sequence
  oligonucleotide BG19062
acacgctacggggagcagcggtatgcacggt

SEQ ID NO: 147 - DNA, 25 bp, Artificial Sequence
  oligonucleotide BG13879
catttttgttctacaaaatgaagca

SEQ ID NO: 148 - DNA, 35 bp, Artificial Sequence
  oligonucleotide BG20456
caacttatatcgtatggagcagcggtatgcacggt

SEQ ID NO: 149 - DNA, 38 bp, Artificial Sequence
  oligonucleotide BG20457
gctacggggagcagcggtaaaaaaaccccgccgaagcg

SEQ ID NO: 150 - DNA, 26 bp, Artificial Sequence
  oligonucleotide BG20239
ggacatcacctctcacaacgaagatt

SEQ ID NO: 151 - DNA, 26 bp, Artificial Sequence
  oligonucleotide BG20240
aatcttcgttgtgagaggtgatgtcc

SEQ ID NO: 152 - DNA, 40 bp, Artificial Sequence
  oligonucleotide BG20514
ccgctgctccatacgatataagttgtaattcggtaccccg

SEQ ID NO: 153 - DNA, 18 bp, Artificial Sequence
  oligonucleotide BG20455
accgctgctccccgtagc

SEQ ID NO: 154 - DNA, 25 bp, Artificial Sequence
  oligonucleotide BG13880
tgcttcattttgtagaacaaaaatg

SEQ ID NO: 155 - DNA, 25 bp, Artificial Sequence
  oligonucleotide BG13879
catttttgttctacaaaatgaagca

SEQ ID NO: 156 - DNA, 35 bp, Artificial Sequence; identical to SEQ ID NO: 148
  oligonucleotide BG20456

SEQ ID NO: 157 - DNA, 38 bp, Artificial Sequence; identical to SEQ ID NO: 149
  oligonucleotide BG20457

SEQ ID NO: 158 - DNA, 26 bp, Artificial Sequence
  oligonucleotide BG20239
ggacatcacctctcacaacgaagatt

SEQ ID NO: 159 - DNA, 26 bp, Artificial Sequence
  oligonucleotide BG20240
aatcttcgttgtgagaggtgatgtcc

SEQ ID NO: 160 - DNA, 40 bp, Artificial Sequence; identical to SEQ ID NO: 152
  oligonucleotide BG20514

SEQ ID NO: 161 - DNA, 74 bp, Artificial Sequence
  oligonucleotide BG20383
cggatcgatgtacacaaccgactgcacccaaacgaacacaaatcttagcatcatagcttcaaaatgtttctact

SEQ ID NO: 162 - DNA, 59 bp, Artificial Sequence
  oligonucleotide BG21049
gccggtacctcctccacccaccttcctcttcttcttgggctttttcttttttgcctggc

SEQ ID NO: 163 - DNA, 41 bp, Artificial Sequence
  oligonucleotide BG21050
ggccaggcaaaaaagaaaaagcccaagaagaagaggaaggt

SEQ ID NO: 164 - DNA, 52 bp, Artificial Sequence
  oligonucleotide BG21051
gggcgtgaatgtaagcgtgacataactaatttatagcatcttgatcttgttc

SEQ ID NO: 165 - DNA, 52 bp, Artificial Sequence
  oligonucleotide BG21052
aatggagagaacaagatcaagatgctataaattagttatgtcacgcttacat

SEQ ID NO: 166 - DNA, 71 bp, Artificial Sequence
  oligonucleotide BG20390
caacaggaggcggatggatatactgtggtctggaagatgccggaaagcgtgatcccaatacaacagatcac

SEQ ID NO: 167 - DNA, 45 bp, Artificial Sequence
  oligonucleotide BG20384
cgatagaacctaaataaaacgagctcaaattaaagccttcgagcg

SEQ ID NO: 168 - DNA, 23 bp, Artificial Sequence
  oligonucleotide BG20385
agaagatttctcttcaatctcct

SEQ ID NO: 169 - DNA, 70 bp, Artificial Sequence
  oligonucleotide BG20386
tgctaagatttgtgttcgtttgggtgcagtcggttgtgtacatcgatccgcccttatcaaggatacctgg

SEQ ID NO: 170 - DNA, 66 bp, Artificial Sequence
  oligonucleotide BG20387
acgctttccggcatcttccagaccacagtatatccatccgcctcctgttgggcgattacacaagcg

SEQ ID NO: 171 - DNA, 17 bp, Artificial Sequence
  oligonucleotide BG20388
tctcctcttcgatgacc

SEQ ID NO: 172 - DNA, 49 bp, Artificial Sequence
  oligonucleotide BG20389
tgggacgctcgaaggctttaatttgagctcgttttatttaggttctatc

SEQ ID NO: 173 - DNA, 23 bp, Artificial Sequence
  oligonucleotide BG21235
aagatggttccgttcaactagtg

SEQ ID NO: 174 - DNA, 23 bp, Artificial Sequence
  oligonucleotide BG21236
tagttgaacggaaccatcttgtc

SEQ ID NO: 175 - DNA, 23 bp, Artificial Sequence
  oligonucleotide BG21237
cccaaagtgttcctgtggaagtg

SEQ ID NO: 176 - DNA, 23 bp, Artificial Sequence
  oligonucleotide BG21238
ttccacaggaacactttggggtc

SEQ ID NO: 177 - DNA, 23 bp, Artificial Sequence
  oligonucleotide BG21239
tgcaaatgcctagaggtgttgtg

SEQ ID NO: 178 - DNA, 23 bp, Artificial Sequence
  oligonucleotide BG21240
aacacctctaggcatttgcagtc

SEQ ID NO: 179 - DNA, 23 bp, Artificial Sequence
  oligonucleotide BG21241
atacaagacaaatatattcagtg

SEQ ID NO: 180 - DNA, 23 bp, Artificial Sequence
  oligonucleotide BG21242
tgaatatatttgtcttgtatgtc

SEQ ID NO: 181 - DNA, 23 bp, Artificial Sequence
  oligonucleotide BG21243
aagcaagaagaagaagttctgtg

SEQ ID NO: 182 - DNA, 23 bp, Artificial Sequence
  oligonucleotide BG21244
agaacttcttcttcttgcttgtc

SEQ ID NO: 183 - DNA, 19 bp, Artificial Sequence
  A motif sequence
  misc_feature (2)..(3): n is a, c, g, or t
  misc_feature (5)..(6): n is a, c, g, or t
  misc_feature (8)..(9): n is a, c, g, or t
  misc_feature (11)..(12): n is a, c, g, or t
  misc_feature (14)..(15): n is a, c, g, or t
  misc_feature (17)..(18): n is a, c, g, or t
annannannannannanna

SEQ ID NO: 184 - DNA, 19 bp, Artificial Sequence
  G motif sequence
  misc_feature (2)..(3): n is a, c, g, or t
  misc_feature (5)..(6): n is a, c, g, or t
  misc_feature (8)..(9): n is a, c, g, or t
  misc_feature (11)..(12): n is a, c, g, or t
  misc_feature (14)..(15): n is a, c, g, or t
  misc_feature (17)..(18): n is a, c, g, or t
gnngnngnngnngnngnng

SEQ ID NO: 185 - DNA, 20 bp, Artificial Sequence
  C-tile target wt
ttcattaaaaattgaattga

SEQ ID NO: 186 - DNA, 20 bp, Artificial Sequence
  C-tile target C-tile 1
ccccccaaaaattgaattga

SEQ ID NO: 187 - DNA, 20 bp, Artificial Sequence
  C-tile target C-tile 2
ttcccccccaattgaattga

SEQ ID NO: 188 - DNA, 20 bp, Artificial Sequence
  C-tile target C-tile 3
ttcattcccccctgaattga

SEQ ID NO: 189 - DNA, 20 bp, Artificial Sequence
  C-tile target C-tile 4
ttcattaaaccccccattga

SEQ ID NO: 190 - DNA, 20 bp, Artificial Sequence
  C-tile target C-tile 5
ttcattaaaaatccccccga

SEQ ID NO: 191 - DNA, 20 bp, Artificial Sequence
  C-tile target C-tile 6
ttcattaaaaattgaccccc

SEQ ID NO: 192 - DNA, 20 bp, Artificial Sequence
  Binding sequence
ttgaattagatggtgatgtt

SEQ ID NO: 193 - DNA, 20 bp, Artificial Sequence
  C1 motif
catcgtcaacgtcagcatct

SEQ ID NO: 194 - DNA, 20 bp, Artificial Sequence
  C2 motif
actgctacagctacgacttc

SEQ ID NO: 195 - DNA, 20 bp, Artificial Sequence
  C3 motif
atcgtcaacgtcagcatcta

SEQ ID NO: 196 - protein, 7 aa, Saccharomyces cerevisiae
TQSVPVE

SEQ ID NO: 197 - protein, 7 aa, Saccharomyces cerevisiae
VQMPRGV

SEQ ID NO: 198 - protein, 7 aa, Saccharomyces cerevisiae
KQEEEVL

SEQ ID NO: 199 - protein, 5 aa, Artificial Sequence
  Base edited ADE2_1
SVPVE

SEQ ID NO: 200 - protein, 5 aa, Artificial Sequence
  Base edited ADE2_2
MPRGV

SEQ ID NO: 201 - protein, 5 aa, Artificial Sequence
  Base edited ADE2_3
EEEVL

SEQ ID NO: 202 - DNA, 20 bp, Saccharomyces cerevisiae
cccaaagtgttcctgtggaa

SEQ ID NO: 203 - DNA, 20 bp, Artificial Sequence
  Base edited ADE2_1
cctaaagtgttcctgtggaa

SEQ ID NO: 204 - DNA, 20 bp, Saccharomyces cerevisiae
tgcaaatgcctagaggtgtt

SEQ ID NO: 205 - DNA, 20 bp, Artificial Sequence
  Base edited ADE2_2
tgtaaatgcctagaggtgtt

SEQ ID NO: 206 - DNA, 20 bp, Saccharomyces cerevisiae
aagcaagaagaagaagttct

SEQ ID NO: 207 - DNA, 20 bp, Artificial Sequence
  Base edited ADE2_3
aagtaagaagaagaagttct

SEQ ID NO: 208 - protein, 166 aa, Artificial Sequence
  TadA8e
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 209 - protein, 184 aa, Artificial Sequence
  TadA8e_eng
NISSQAKKQANFYRMPRQSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 210 - DNA, 23 bp, Artificial Sequence
  A1 motif
accactatcagcacgagtatggt

SEQ ID NO: 211 - DNA, 23 bp, Artificial Sequence
  A2 motif
cacaattacgactattaggaggt

SEQ ID NO: 212 - DNA, 23 bp, Artificial Sequence
  A3 motif
ccaatattagcatgatcagcggt

SEQ ID NO: 213 - DNA, 23 bp, Artificial Sequence
  C1 motif
catcgtcaacgtcagcatctggt

SEQ ID NO: 214 - DNA, 23 bp, Artificial Sequence
  C2 motif
actcctacagctacgacttcggt

SEQ ID NO: 215 - DNA, 23 bp, Artificial Sequence
  C3 motif
atcctcaacgtcagcatctaggt

SEQ ID NO: 216 - DNA, 20 bp, Artificial Sequence
  C1 motif
catcgtcaacgtcagcatct

SEQ ID NO: 217 - DNA, 20 bp, Artificial Sequence
  C2 motif
actcctacagctacgacttc

SEQ ID NO: 218 - DNA, 20 bp, Artificial Sequence
  C3 motif
atcctcaacgtcagcatcta

SEQ ID NO: 219 - DNA, 20 bp, Artificial Sequence
  A1 motif
accactatcagcacgagtat

SEQ ID NO: 220 - DNA, 20 bp, Artificial Sequence
  A2 motif
cacaattacgactattagga

SEQ ID NO: 221 - DNA, 20 bp, Artificial Sequence
  A3 motif
ccaatattagcatgatcagc

SEQ ID NO: 222 - protein, 849 aa, Artificial Sequence
  Cas12f1 CBE1
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIALGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIKGGGGSGGGGSAEYVRALFDFNGNDEEDLPFKKGDILRIRDKPEEQWWNAEDSEGKRGMIPVPYVEKYSGDYKDHDGDYKDHDIDYKDDDDKSRMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 223 - protein, 772 aa, Artificial Sequence
  Cas12f1 CBE2
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIALGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIKSGSETPGTSESATPESMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 224 - DNA, 20 bp, Artificial Sequence
  C1 tile
catcgtcaacgtcagcatct

SEQ ID NO: 225 - DNA, 20 bp, Artificial Sequence
  C2 tile
actcctacagctacgacttc

SEQ ID NO: 226 - DNA, 20 bp, Artificial Sequence
  C3 tile
atcctcaacgtcagcatcta

SEQ ID NO: 227 - DNA, 20 bp, Artificial Sequence
  G1 tile
gtagcagttgcagtcgtaga

SEQ ID NO: 228 - DNA, 20 bp, Artificial Sequence
  G2 tile
tgaggatgtcgatgctgaag

SEQ ID NO: 229 - DNA, 20 bp, Artificial Sequence
  G3 tile
taggagttgcagtcgtagat